SEQUENCE LISTING (24 sequences)

SEQ ID NO: 1 - DNA, 47 bp, Artificial Sequence
  Synthetic oligonucleotide
actcctcaacgcacgaaacacgaccgaatatcctgcattcccgaacc

SEQ ID NO: 2 - DNA, 48 bp, Artificial Sequence
  Synthetic oligonucleotide
actcctcaacgcacgaaacacgacccttcaaaaggccacctgttactg

SEQ ID NO: 3 - DNA, 45 bp, Artificial Sequence
  Synthetic oligonucleotide
actcctcaacgcacgaaacacgacctccttgggtccctgtagcag

SEQ ID NO: 4 - DNA, 48 bp, Artificial Sequence
  Synthetic oligonucleotide
actcctcaacgcacgaaacacgaccgtgtcttctgcttgattcctctg

SEQ ID NO: 5 - DNA, 46 bp, Artificial Sequence
  Synthetic oligonucleotide
actcctcaacgcacgaaacacgacctggcagcgacatggtttgttg

SEQ ID NO: 6 - DNA, 48 bp, Artificial Sequence
  Synthetic oligonucleotide
actcctcaacgcacgaaacacgaccgcaattatggtttctccgccaag

SEQ ID NO: 7 - DNA, 51 bp, Artificial Sequence
  Synthetic oligonucleotide
cagcacaggaaccaaccacagcaataagggagactttgcgatgtacttgac

SEQ ID NO: 8 - DNA, 25 bp, Artificial Sequence
  Synthetic oligonucleotide
actcctcaacgcacgaaacacgacc

SEQ ID NO: 9 - DNA, 32 bp, Artificial Sequence
  Synthetic oligonucleotide
aaaacagcacaggaaccaaccacagcaanaag

SEQ ID NO: 10 - DNA, 44 bp, Artificial Sequence
  Synthetic oligonucleotide
actcctcaacgcacgaaacacgacccgtcgctggcgtgcgttcc

SEQ ID NO: 11 - DNA, 48 bp, Artificial Sequence
  Synthetic oligonucleotide
actcctcaacgcacgaaacacgacccctttaccgctgattcgtggaac

SEQ ID NO: 12 - DNA, 46 bp, Artificial Sequence
  Synthetic oligonucleotide
actcctcaacgcacgaaacacgaccggttagggggtaaatcccggc

SEQ ID NO: 13 - DNA, 50 bp, Artificial Sequence
  Synthetic oligonucleotide
actcctcaacgcacgaaacacgaccggcctgtatagcttcagtgattgcg

SEQ ID NO: 14 - DNA, 48 bp, Artificial Sequence
  Synthetic oligonucleotide
actcctcaacgcacgaaacacgaccgctgtcagaggcttgtgtttgtg

SEQ ID NO: 15 - DNA, 47 bp, Artificial Sequence
  Synthetic oligonucleotide
actcctcaacgcacgaaacacgacccgtagcgatcaagccatgaatg

SEQ ID NO: 16 - DNA, 47 bp, Artificial Sequence
  Synthetic oligonucleotide
actcctcaacgcacgaaacacgacccttcaagtggagcatcaggcag

SEQ ID NO: 17 - DNA, 46 bp, Artificial Sequence
  Synthetic oligonucleotide
actcctcaacgcacgaaacacgaccgtatccattgagcattgccgc

SEQ ID NO: 18 - DNA, 49 bp, Artificial Sequence
  Synthetic oligonucleotide
actcctcaacgcacgaaacacgaccggaatgcatcgctcagtgttgatc

SEQ ID NO: 19 - DNA, 46 bp, Artificial Sequence
  Synthetic oligonucleotide
actcctcaacgcacgaaacacgacccgtcagccgtaagtcttgatc

SEQ ID NO: 20 - DNA, 47 bp, Artificial Sequence
  Synthetic oligonucleotide
actcctcaacgcacgaaacacgaccgccaacatggtgatgattctgc

SEQ ID NO: 21 - DNA, 48 bp, Artificial Sequence
  Synthetic oligonucleotide
actcctcaacgcacgaaacacgaccctcgttgcccggtaacaacagcc

SEQ ID NO: 22 - DNA, 48 bp, Artificial Sequence
  Synthetic oligonucleotide
actcctcaacgcacgaaacacgacccgacataaagatatccatctacg

SEQ ID NO: 23 - DNA, 45 bp, Artificial Sequence
  Synthetic oligonucleotide
actcctcaacgcacgaaacacgacccaatatgcaatgctgttggg

SEQ ID NO: 24 - DNA, 32 bp, Artificial Sequence
  Synthetic oligonucleotide
aaaacagcacaggaaccaaccacagcaataag

(19) United States
(12) Patent Application Publication
(10) Pub. No.: US 2011/0269194 A1
(43) Pub. Date: Nov. 3, 2011

(54) MATERIALS AND METHODS FOR NUCLEIC ACID FRACTIONATION BY SOLID PHASE ENTRAPMENT AND ENZYME-MEDIATED DETACHMENT

(75) Inventor: VLADIMIR MAKAROV, ANN ARBOR, MI (US)

(73) Assignee: SWIFT BIOSCIENCES, INC., ANN ARBOR, MI (US)

(21) Appl. No.: 13/090,966

(22) Filed: Apr. 20, 2011

Related U.S. Application Data
(60) Provisional application No. 61/325,982, filed on Apr. 20, 2010.

(51) Int. Cl. C12P 19/34; C12S 99/00; C07H 21/04; C12N 9/96

(52) U.S. Cl. 435/91.53; 435/188; 435/91.5; 435/262; 536/23.1

(57) ABSTRACT

Materials and methods are provided for the gel-free fractionation of polynucleotide molecules. According to the present invention, fractionation is size-based or sequence-based.

This application claims benefit to U.S. Provisional Application Ser. No. 61/325,982, filed Apr. 20, 2010, which is incorporated herein by reference in their entirety.

This application contains, as a separate part of disclosure, a Sequence Listing in computer-readable form (filename: 43311A_SeqListing.txt; created Apr. 18, 2011; 5,826 bytes—ASCII text file) which is incorporated by reference in its entirety.

### BACKGROUND

Since the discovery of DNA, size analyses of nucleic acids have been important tools to characterize and determine DNA structure and isolate nucleic acid molecules (plasmids, viruses, PCR products, etc.) for gene cloning and other biotechnology applications. Recently, the need for isolation of DNA fragments of a specific size has been in high demand due to various microarray applications and, most significantly, due to development of novel, highly parallel and massive methods of DNA sequence analysis (frequently referred to as next generation sequencing (NGS) methods).

Numerous methods exist for fractionating polynucleotides based on size or sequence. Current methods of DNA or RNA size fractionation typically involve five steps: (1) size separations by gel agarose or acrylamide gel; (2) gel staining; (3) dissection of the gel area containing appropriate size fraction; (4) extraction of DNA/RNA from the gel; and (5) purification of the extracted nucleic acid. Gel-based DNA or RNA size fractionation have many limitations. For example, current fractionation methods are typically labor-intensive, have long run-times, are low throughput, and are not amenable to automation.

For sequence-specific fractionation of polynucleotides, customized oligonucleotide capture arrays, long distance PCR, small volume/large scale PCR, and soluble RNA capture probes are frequently employed. Like the limitations of the current size-based fractionation methods, many of these sequence-specific methods are generally associated with high cost, long protocol times, and sequence bias, and these methods are not amenable to automation and can not be used to deplete (as opposed to enrichment) sequences or to enrich rare, mutated alleles.

Thus, there exists a need in the art to provide materials and methods that address these aforementioned limitations.

### SUMMARY OF THE INVENTION

The present disclosure provides materials and methods for nucleic acid fractionation by Solid Phase Entrapment and Enzyme-mediated Detachment (SPEED).

In one aspect of the disclosure, a method of preparing a desired polynucleotide is provided comprising contacting a labeled polynucleotide having a double-stranded region comprising a first strand and a second strand wherein either the first strand or second strand is labeled and immobilized with an enzyme or enzyme mixture under conditions wherein the interaction of the first strand with the second strand is reduced, thereby resulting in dissociation of the second strand from the first strand, wherein either an unlabeled first strand or unlabeled second strand is the desired polynucleotide.

In one aspect of the method, the first strand has a 5′ terminus and a 3′ terminus and the second strand has a 5′ terminus and a 3′ terminus; and wherein the labeled polynucleotide comprises a double-stranded target sequence between a double-stranded 5′ terminal sequence located proximal to the label, and a double-stranded 3′ terminal sequence located distal to the label; the 5′ terminal sequence including the 5′ terminus of the first strand and a sequence adjacent and the 3′ terminus of the second strand and a sequence adjacent; the 3′ terminal sequence including the 3′ terminus of the first strand and a sequence adjacent and the 5′ terminus of the second strand and a sequence adjacent.

In another aspect of the method, the desired polynucleotide is a polynucleotide of a desired size.

In various aspects of the invention, the enzyme or enzyme mixture is capable of catalyzing nick-translation polymerization, capable of catalyzing strand-displacement polymerization, capable of catalyzing double-stranded polynucleotide-specific degradation, or capable of catalyzing polynucleotide strand unwinding.

In still another aspect of the method, the enzyme or enzyme mixture contacts the labeled polynucleotide from an end distal to the label. In another aspect, the enzyme or enzyme mixture contacts the labeled polynucleotide at both ends of the labeled polynucleotide. In still another aspect, the contacting of the labeled polynucleotide with the enzyme or enzyme mixture precedes immobilization of the labeled strand.

As will be readily appreciated by a person of skill in the art, depending on the type of enzyme used in the methods disclosed herein, dissociated polynucleotides are single-stranded or double-stranded. Thus, in one aspect of the above method, the dissociated polynucleotide is double-stranded.

In yet another aspect, the method further comprises the step of isolating the dissociated polynucleotide. In another aspect, the method further comprises the step of amplifying the isolated polynucleotide.

In still another aspect of the method, the labeled polynucleotide is prepared following random fragmentation by sonication, nebulization, chemical, physical or enzymatic treatment of genomic DNA. In another aspect, the labeled polynucleotide is prepared following cleavage of genomic DNA by a restriction endonuclease. In another aspect, the labeled polynucleotide is prepared following PCR of one or several genomic regions. In yet another aspect, the labeled polynucleotide is prepared following whole genome amplification (WGA) or whole transcriptome amplification (WTA) of genomic DNA or RNA. In another aspect, the labeled polynucleotide is prepared by chemical or enzymatic synthesis. In still another aspect, the labeled polynucleotide is further prepared following attachment to terminal adaptor sequences.

In another aspect of the method the enzyme is a DNA polymerase. In various aspects, the DNA polymerase is selected from the group consisting of nick-translating DNA polymerases: DNA polymerase I, Taq DNA polymerase, full-size Bst DNA polymerase, DyNAzyme, Thermus caldophilus DNA polymerase, and Thermus brockiamus DNA polymerase. In other various aspects, the DNA polymerase is selected from the group consisting of strand-displacing DNA polymerases: Klenow fragment of DNA polymerase I (exo-), reverse transcriptase, Sequenase, Phi 29 DNA polymerase, Bst DNA polymerase, DisplaceAce DNA polymerase, Vent DNA polymerase (exo-), and Deep Vent DNA polymerase (exo-).

In one aspect of the method, immobilizing attaches the 5′ terminus of the first strand to a support.

In another aspect, the method further comprises the step of introducing a non-extendable nick or gap at the 5′ terminal sequence on the first strand proximal to the solid support. In another aspect, the method further comprises the step of protecting the 5′ terminal sequence on the first strand from 5′ exonuclease activity by introducing one or more nuclease-resistant bases in the gap.

In yet another aspect of the method, the 5′ terminal sequence of the first strand comprises nuclease-resistant bases.

In one the method further comprises the step of introducing an extendable nick or gap at the 3′ terminal sequence on the second strand distal to the solid support.

In another aspect of the method, immobilizing attaches the 3′ terminus of the second strand to a support. In still another aspect, the method further comprises the step of introducing a nick or gap at the 5′ end of the terminal sequence on the first strand proximal to the solid support.

In yet another aspect, the method further comprises the step of introducing a nick or gap at the 5′ end of the terminal sequence on the second strand distal to the solid support, thereby creating an extendable break in the second strand of the labeled polynucleotide.

In one aspect of the method, the enzyme is an exonuclease. In various aspects, the exonuclease is selected from the group consisting of a 5′-exonuclease and 3′-exonuclease. In still other aspects, the 5′-exonuclease is selected from the group consisting of lambda exonuclease and T7 gene 6 exonuclease, and the 3′-exonuclease is exonuclease III. In another aspect, the 3′-exonuclease is a “proofreading” DNA polymerase with 3′-5′ exonuclease activity. In another aspect, the dissociated polynucleotide is single-stranded.

In another aspect of the method, immobilizing attaches the 5′ terminus of the first strand to a support. In yet another aspect, the method further comprises the step of introducing a nick or gap at the 5′ terminal sequence on the first strand proximal to the solid support. In still another aspect, the method further comprises the step of protecting the 5′ terminal sequence on the first strand from 5′ exonuclease activity by introducing one or more nuclease-resistant bases in the gap.

In another aspect of the method, the 5′ terminal sequence of the first strand comprises nuclease-resistant bases. In yet another aspect, the method further comprises the step of protecting the 5′ terminal sequence on the second strand from 3′ exonuclease activity by introducing one or more nuclease-resistant bases in the gap.

In yet another aspect of the method, the 5′ terminal sequence of the second strand comprises nuclease-resistant bases.

In still another aspect of the invention, immobilizing attaches the 3′ terminus of the second strand to a support. In yet another aspect, the method further comprises the step of introducing a nick or gap at the 5′ terminal sequence on the second strand proximal to the solid support. In still another aspect, the method further comprises the step of protecting the 5′ terminal sequence on the second strand from 3′ exonuclease activity by introducing one or more nuclease-resistant bases in the gap.

In another aspect of the method, the 5′ terminal sequence of the second strand comprises nuclease-resistant bases. In yet another aspect, the method further comprises the step of protecting the 5′ terminal sequence on the first strand from 5′ exonuclease activity by introducing one or more nuclease-resistant bases in the gap.

In still another aspect of the method, the 5′ terminal sequence of the first strand comprises nuclease-resistant bases.

In another aspect of the method, the enzyme is a helicase. In various aspects, the helicase is selected from the group consisting of a 5′-3′ helicase and a 3′-5′ helicase. In another aspect, the dissociated polynucleotide is single-stranded. In still another aspect, immobilizing attaches the 5′ terminus of the first strand to a support. In another aspect, immobilizing attaches the 3′ terminus of the second strand to a support.

In yet another aspect, the method further comprises the step of introducing a 5′ single-stranded overhang on the 5′ terminus of the second strand. In a specific aspect, the enzyme with helicase activity is an enzyme with 5′ to 3′ helicase activity.

In another aspect, the method further comprises the step of introducing a 3′ single-stranded overhang on the 3′ terminus of the first strand. In a specific aspect, the enzyme with helicase activity is an enzyme with 3′ to 5′ helicase activity.

In addition to preparing polynucleotides of a desired size, polynucleotides of a desired sequence are also prepared according to numerous aspects of the present disclosure. Thus, in aspect of the disclosure, the desired polynucleotide is a polynucleotide of desired sequence.

In another aspect of the method, the first strand has a 5′ terminus and a 3′ terminus and the second strand has a 5′ terminus and a 3′ terminus; and wherein the labeled polynucleotide comprises a double-stranded target sequence between a double-stranded 5′ terminal sequence located proximal to the label, and a single-stranded 3′ terminal sequence located distal to the label; the 5′ terminal sequence including the 5′ terminus of the first strand and a sequence adjacent and the 3′ terminus of the second strand and a sequence adjacent; the 3′ terminal sequence including the 3′ terminus of the first strand. In another aspect, immobilizing attaches the 5′ terminus of the first strand to a support.

In still another aspect of the method, the enzyme is capable of catalyzing nick-translation polymerization. In another aspect, the enzyme is capable of catalyzing strand-displacement polymerization.

In another aspect of the method, the double-stranded region of the labeled polynucleotide is prepared by the steps of (a) hybridizing a first target-specific primer to the first strand of the labeled polynucleotide, and (b) synthesizing the second strand from the first target-specific primer to the 5′ terminus of the first strand, wherein the 5′ terminus of the first strand comprises one of more RNA residues or a nickase endonuclease site.

In another aspect, the double-stranded region of the labeled polynucleotide is prepared by hybridizing a polynucleotide complementary to the 5′ terminus of the first strand, wherein the 5′ terminus of the first strand comprises one or more RNA residues or a nickase endonuclease site. In yet another aspect, the method further comprises the step of introducing a non-extendable nick or gap in the 5′ terminus of the first strand. In another aspect, the non-extendable nick or gap is introduced by (i) removing the one or more RNA residues, and (ii) introducing a dideoxynucleotide. In another aspect, the non-extandable nick is introduced by (i) nicking reaction using a nickase enzyme, and (ii) introducing a dideoxynucleotide. In yet another aspect, the method further comprises the steps of (a) hybridizing a second primer to the first strand, the second primer selected from the group consisting of (i) a second target-specific primer, and (ii) a universal primer, and (b) releasing the polynucleotide of desired sequence into solution by synthesizing a new second strand from the second primer to the 5′ terminus of the first strand.

In another aspect of the method, the labeled polynucleotide comprises a first strand having a 5′ terminus and a 3′ terminus and a second strand having a 5′ terminus and a 3′ terminus is contacted with an enzyme with polymerase activity under conditions wherein a new second strand is synthesized. In another aspect, the labeled polynucleotide is immobilized at the 3′ terminus of the second strand to a support. In still another aspect, the double-stranded region of the labeled polynucleotide is prepared by hybridizing a labeled probe to the first strand of the labeled polynucleotide. In yet another aspect, the method further comprises the steps of (a) hybridizing a primer to the first strand, and (b) synthesizing a new second strand from the primer to a location 3′ of the double-stranded region.

In one aspect of the method, the desired polynucleotide is partially double-stranded.

In another aspect of the method, the labeled polynucleotide comprises label selected from the group consisting of: biotin, streptavidin, avidin, and digoxigenin.

In still another aspect of the method, the separation of the desired polynucleotide is regulated by time of enzyme activity.

Compositions are also provided by the present disclosure. In one aspect, a composition is provided comprising a labeled polynucleotide having a double-stranded region, the labeled polynucleotide comprising a first strand having a 5′ terminus and a 3′ terminus and a second strand having a 5′ terminus and a 3′ terminus, the labeled polynucleotide further comprising a double-stranded target sequence between a double-stranded 5′ terminal sequence and a double-stranded 3′ terminal sequence, the 5′ terminal sequence including the 5′ terminus of the first strand and a sequence adjacent and the 3′ terminus of the second strand and a sequence adjacent, the 3′ terminal sequence including the 3′ terminus of the first strand and a sequence adjacent and the 5′ terminus of the second strand and a sequence adjacent, the 5′ terminal sequence comprising one or more nuclease-resistant bases or one or more RNA residues. In another aspect, a composition is provided comprising a labeled polynucleotide having a double-stranded region, the labeled polynucleotide comprising a first strand having a 5′ terminus and a 3′ terminus and a second strand having a 5′ terminus and a 3′ terminus, the labeled polynucleotide further comprising a double-stranded target sequence between a double-stranded 5′ terminal sequence and a single stranded 3′ terminal sequence, the 5′ terminal sequence including the 5′ terminus of the first strand and a sequence adjacent and the 3′ terminus of the second strand and a sequence adjacent, the 3′ terminal sequence including the 3′ terminus of the first strand and a sequence adjacent and the 5′ terminus of the second strand and a sequence adjacent, the 5′ terminal sequence comprising one or more nuclease-resistant bases or one or more RNA residues.

Additional methods of preparing polynucleotides of a desired characteristic (e.g., size and/or sequence) are also provided. In one aspect, a method of preparing a desired polynucleotide is provided comprising the steps of: a) preparing a labeled polynucleotide, wherein the labeled polynucleotide comprises a first strand having a 5′ terminus and a 3′ terminus and a second strand having a 5′ terminus and a 3′ terminus, and wherein the labeled polynucleotide comprises a double-stranded target sequence between a double-stranded 5′ terminal sequence located proximal to the label, and a double-stranded 3′ terminal sequence located distal to the label, the double-stranded 5′ terminal sequence including the 5′ terminus of the first strand and a sequence adjacent and the 3′ terminus of the second strand and a sequence adjacent, the double-stranded 3′ terminal sequence including the 3′ terminus of the first strand and a sequence adjacent and the 5′ terminus of the second strand and a sequence adjacent; b) immobilizing the labeled polynucleotide, said immobilizing comprising attaching the 5′ end of the double-stranded 5′ terminal sequence on the first strand to a solid phase binding partner or, in the alternative, attaching the 3′ end of the double-stranded 5′ terminal sequence on the second strand to a solid phase binding partner; c) optionally introducing a nick or gap at the 5′ end of the double-stranded 5′ terminal sequence on the first strand proximal to the solid phase binding partner in the case of 5′ end attachment in step b); d) introducing a nick or gap at the 5′ end of the double-stranded 3′ terminal sequence on the second strand distal to the solid phase binding partner; thereby creating an extendable break in the 5′ end of the double-stranded 3′ terminal sequence on the second strand distal to the solid phase binding partner; and f) contacting the labeled polynucleotide with an enzyme with 5′-3′ exonuclease and polymerase activity under conditions wherein the second strand of the labeled polynucleotide is degraded up to a non-degradable location and a new second strand is synthesized up to a non-extendable location, at which point the desired polynucleotide is separated from the labeled polynucleotide.

In another aspect, a method of preparing a desired polynucleotide is provided comprising the steps of: a) preparing a labeled polynucleotide, wherein the labeled polynucleotide comprises a first strand having a 5′ terminus and a 3′ terminus and a second strand having a 5′ terminus and a 3′ terminus, and wherein the labeled polynucleotide comprises a double-stranded target sequence between a double-stranded 5′ terminal sequence located proximal to the label, and a double-stranded 3′ terminal sequence located distal to the label, the double-stranded 5′ terminal sequence including the 5′ terminus of the first strand and a sequence adjacent and the 3′ terminus of the second strand and a sequence adjacent, the double-stranded 3′ terminal sequence including the 3′ terminus of the first strand and a sequence adjacent and the 5′ terminus of the second strand and a sequence adjacent; b) immobilizing the labeled polynucleotide, said immobilizing comprising attaching the 5′ end of the double-stranded 5′ terminal sequence on the first strand to a solid phase binding partner or, in the alternative, attaching the 3′ end of the double-stranded 5′ terminal sequence on the second strand to a solid phase binding partner; c) introducing a nick or gap at the 5′ end of the double-stranded 5′ terminal sequence on the first strand proximal to the solid phase binding partner; d) introducing a nick or gap at the 5′ end of the double-stranded 3′ terminal sequence on the second strand distal to the solid phase binding partner; and e) contacting the labeled polynucleotide with an enzyme with 5′-3′ exonuclease and polymerase activity under conditions wherein the first strand and the second strand of the labeled polynucleotide are degraded, and a new first strand and a new second strand are synthesized, up to an enzyme collision location, at which point the desired polynucleotide is separated from the labeled polynucleotide.

In still another aspect, a method of preparing a desired polynucleotide is provided comprising the steps of: a) preparing a labeled polynucleotide, wherein the labeled polynucleotide comprises a first strand having a 5′ terminus and a 3′ terminus and a second strand having a 5′ terminus and a 3′ terminus, and wherein the labeled polynucleotide comprises a double-stranded target sequence between a double-stranded 5′ terminal sequence located proximal to the label, and a double-stranded 3′ terminal sequence located distal to the label, the double-stranded 5′ terminal sequence including the 5′ terminus of the first strand and a sequence adjacent and the 3′ terminus of the second strand and a sequence adjacent, the double-stranded 3′ terminal sequence including the 3′ terminus of the first strand and a sequence adjacent and the 5′ terminus of the second strand and a sequence adjacent; b) immobilizing the labeled polynucleotide, said immobilizing comprising attaching the 5′ end of the double-stranded 5′ terminal sequence on the first strand to a solid phase binding partner or, in the alternative, attaching the 3′ end of the double-stranded 5′ terminal sequence on the second strand to a solid phase binding partner; c) optionally introducing a nick or gap at the 5′ end of the double-stranded 5′ terminal sequence on the first′ strand proximal to the solid phase binding partner in the case of 5′ end attachment in step b); d) introducing nick or gap at the 5′ end of the double-stranded 3′ terminal sequence on the second′ strand distal to the solid phase binding partner; and e) contacting the labeled polynucleotide with an enzyme with polymerase activity under conditions wherein the second strand of the labeled polynucleotide is displaced, and a new second strand is synthesized up to a non-extendable location, at which point the desired polynucleotide is separated from the labeled polynucleotide.

In yet another aspect, a method of preparing a desired polynucleotide comprising the steps of: a) preparing a labeled polynucleotide, wherein the labeled polynucleotide comprises a first strand having a 5′ terminus and a 3′ terminus and a second strand having a 5′ terminus and a 3′ terminus, and wherein the labeled polynucleotide comprises a double-stranded target sequence between a double-stranded 5′ terminal sequence located proximal to the label, and a double-stranded 3′ terminal sequence located distal to the label, the double-stranded 5′ terminal sequence including the 5′ terminus of the first strand and a sequence adjacent and the 3′ terminus of the second strand and a sequence adjacent, the double-stranded 3′ terminal sequence including the 3′ terminus of the first strand and a sequence adjacent and the 5′ terminus of the second strand and a sequence adjacent; b) immobilizing the labeled polynucleotide, said immobilizing comprising attaching the 5′ end of the double-stranded 5′ terminal sequence on the first strand to a solid phase binding partner or attaching the 3′ end of the double-stranded 5′ terminal sequence on the second strand to a solid phase binding partner; c) introducing nick or gap at the 5′ end of the double-stranded 5′ terminal sequence on the first strand proximal to the solid phase binding partner; d) introducing nick or gap at the 5′ end of the double-stranded 3′ terminal sequence on the second strand distal to the solid phase binding partner; and e) contacting the labeled polynucleotide with an enzyme with polymerase activity under conditions wherein the first strand and second strand of the labeled polynucleotide are displaced, and a new first strand and a new second strand are synthesized up to an enzyme collision location, at which point the desired polynucleotide is separated from the labeled polynucleotide.

In another aspect, a method of preparing a desired polynucleotide is provided comprising the steps of: a) preparing a labeled polynucleotide, wherein the labeled polynucleotide comprises a first strand having a 5′ terminus and a 3′ terminus and a second strand having a 5′ terminus and a 3′ terminus, and wherein the labeled polynucleotide comprises a double-stranded target sequence between a double-stranded 5′ terminal sequence located proximal to the label, and a double-stranded 3′ terminal sequence located distal to the label, the double-stranded 5′ terminal sequence including the 5′ terminus of the first strand and a sequence adjacent and the 3′ terminus of the second strand and a sequence adjacent, the double-stranded 3′ terminal sequence including the 3′ terminus of the first strand and a sequence adjacent and the 5′ terminus of the second strand and a sequence adjacent; b) immobilizing the labeled polynucleotide, said immobilizing comprising attaching the 5′ end of the double-stranded 5′ terminal sequence on the first strand to a solid phase binding partner or, in the alternative, attaching the 3′ end of the double-stranded 5′ terminal sequence on the second strand to a solid phase binding partner; c) introducing a nick or gap at the 5′ end of the double-stranded 5′ terminal sequence on the first strand proximal to the solid phase binding partner; d) introducing nuclease-resistant bases at the 5′ end of the double-stranded 5′ terminal sequence on the first strand proximal to the solid phase binding partner, thereby creating a non-degradable break in the 5′ end of the double-stranded 5′ terminal sequence on the first strand proximal to the solid phase binding partner; and e) contacting the labeled polynucleotide with an enzyme with 5′-3′ exonuclease activity under conditions wherein the second strand of the labeled polynucleotide is degraded up to a non-degradable location, at which point the desired polynucleotide is separated from the labeled polynucleotide.

In still another aspect, a method of preparing a desired polynucleotide comprising the steps of: a) preparing a labeled polynucleotide, wherein the labeled polynucleotide comprises a first strand having a 5′ terminus and a 3′ terminus and a second strand having a 5′ terminus and a 3′ terminus, and wherein the labeled polynucleotide comprises a double-stranded target sequence between a double-stranded 5′ terminal sequence located proximal to the label, and a double-stranded 3′ terminal sequence located distal to the label, the double-stranded 5′ terminal sequence including the 5′ terminus of the first strand and a sequence adjacent and the 3′ terminus of the second strand and a sequence adjacent, the double-stranded 3′ terminal sequence including the 3′ terminus of the first strand and a sequence adjacent and the 5′ terminus of the second strand and a sequence adjacent; b) immobilizing the labeled polynucleotide, said immobilizing comprising attaching the 5′ end of the double-stranded 5′ terminal sequence on the first strand to a solid phase binding partner or, in the alternative, attaching the 3′ end of the double-stranded 5′ terminal sequence on the second strand to a solid phase binding partner; c) introducing nick or gap at the 5′ end of the double-stranded 5′ terminal sequence on the first strand proximal to the solid phase binding partner; and d) contacting the dsDNA fragment with an enzyme with 5′-3′ exonuclease activity under conditions wherein the first strand and the second strand of the labeled polynucleotide are degraded up to an enzyme collision location, at which point the desired polynucleotide is separated from the labeled polynucleotide.

In yet another aspect, a method of preparing a desired polynucleotide comprising the steps of: a) preparing a labeled polynucleotide, wherein the labeled polynucleotide comprises a first strand having a 5′ terminus and a 3′ terminus and a second strand having a 5′ terminus and a 3′ terminus, and wherein the labeled polynucleotide comprises a double-stranded target sequence between a double-stranded 5′ terminal sequence located proximal to the label, and a double-stranded 3′ terminal sequence located distal to the label, the double-stranded 5′ terminal sequence including the 5′ terminus of the first strand and a sequence adjacent and the 3′ terminus of the second strand and a sequence adjacent, the double-stranded 3′ terminal sequence including the 3′ terminus of the first strand and a sequence adjacent and the 5′ terminus of the second strand and a sequence adjacent; b) immobilizing the labeled polynucleotide, said immobilizing comprising attaching the 5′ end of the double-stranded 5′ terminal sequence on the first strand to a solid phase binding partner or, in the alternative, attaching the 3′ end of the double-stranded 5′ terminal sequence on the second strand to a solid phase binding partner; c) introducing nick or gap at the 3′ end of the double-stranded 5′ terminal sequence on the second strand proximal to the solid phase binding partner; d) introducing nuclease-resistant bases at the 3′ end of the double-stranded 5′ terminal sequence on the second strand proximal to the solid phase binding partner, thereby creating a non-degradable break in the 3′ end of the double-stranded 5′ terminal sequence on the second strand proximal to the solid phase binding partner; and e) contacting the dsDNA fragment with an enzyme with 3′-5′ exonuclease activity under conditions wherein the first strand of the labeled polynucleotide is degraded up to a non-degradable location, at which point the desired polynucleotide is separated from the labeled polynucleotide.

In another aspect, a method of preparing a desired polynucleotide comprising the steps of: a) preparing a labeled polynucleotide, wherein the labeled polynucleotide comprises a first strand having a 5′ terminus and a 3′ terminus and a second strand having a 5′ terminus and a 3′ terminus, and wherein the labeled polynucleotide comprises a double-stranded target sequence between a double-stranded 5′ terminal sequence located proximal to the label, and a double-stranded 3′ terminal sequence located distal to the label, the double-stranded 5′ terminal sequence including the 5′ terminus of the first strand and a sequence adjacent and the 3′ terminus of the second strand and a sequence adjacent, the double-stranded 3′ terminal sequence including the 3′ terminus of the first strand and a sequence adjacent and the 5′ terminus of the second strand and a sequence adjacent; b) immobilizing the labeled polynucleotide, said immobilizing comprising attaching the 5′ end of the double-stranded 5′ terminal sequence on the first strand to a solid phase binding partner or, in the alternative, attaching the 3′ end of the double-stranded 5′ terminal sequence on the second strand to a solid phase binding partner; c) introducing nick or gap at the 3′ end of the double-stranded 5′ terminal sequence on the second strand proximal to the solid phase binding partner; and d) contacting the dsDNA fragment with an enzyme with 3′-5′ exonuclease activity under conditions wherein the first strand and the second strand of the labeled polynucleotide are degraded up to an enzyme collision location, at which point the desired polynucleotide is separated from the labeled polynucleotide.

In another aspect, the above methods are provided wherein immobilizing the labeled polynucleotide occurs after contacting the dsDNA fragment with the enzyme.

In another aspect, a method of preparing a desired polynucleotide comprising the steps of: a) preparing a labeled polynucleotide, wherein the labeled polynucleotide comprises a first strand having a 5′ terminus and a 3′ terminus and a second strand having a 5′ terminus and a 3′ terminus, and wherein the labeled polynucleotide comprises a double-stranded target sequence between a double-stranded 5′ terminal sequence located proximal to the label, and a double-stranded 3′ terminal sequence located distal to the label, the double-stranded 5′ terminal sequence including the 5′ terminus of the first strand and a sequence adjacent and the 3′ terminus of the second strand and a sequence adjacent, the double-stranded 3′ terminal sequence including the 3′ terminus of the first strand and a sequence adjacent and the 5′ terminus of the second strand and a sequence adjacent; b) immobilizing the labeled polynucleotide, said immobilizing comprising attaching the 5′ end of the double-stranded 5′ terminal sequence on the first strand to a solid phase binding partner or, in the alternative, attaching the 3′ end of the double-stranded 5′ terminal sequence on the second strand to a solid phase binding partner; c) introducing a 5′ single-stranded overhang at the second strand of the double-stranded 3′ terminal sequence distal to the label; and d) contacting the single-stranded overhang with an enzyme with 5′-3′ helicase activity under conditions wherein the first strand of the labeled polynucleotide is separated from the second strand up to the end proximal to the solid phase binding partner, at which point the desired polynucleotide is separated from the labeled polynucleotide.

In still another aspect, a method of preparing a desired polynucleotide comprising the steps of: a) preparing a labeled polynucleotide, wherein the labeled polynucleotide comprises a first strand having a 5′ terminus and a 3′ terminus and a second strand having a 5′ terminus and a 3′ terminus, and wherein the labeled polynucleotide comprises a double-stranded target sequence between a double-stranded 5′ terminal sequence located proximal to the label, and a double-stranded 3′ terminal sequence located distal to the label, the double-stranded 5′ terminal sequence including the 5′ terminus of the first strand and a sequence adjacent and the 3′ terminus of the second strand and a sequence adjacent, the double-stranded 3′ terminal sequence including the 3′ terminus of the first strand and a sequence adjacent and the 5′ terminus of the second strand and a sequence adjacent; b) immobilizing the labeled polynucleotide, said immobilizing comprising attaching the 5′ end of the double-stranded 5′ terminal sequence on the first strand to a solid phase binding partner or, in the alternative, attaching the 3′ end of the double-stranded 5′ terminal sequence on the second strand to a solid phase binding partner; c) introducing a 3′ single-stranded overhang at the first strand of the double-stranded 3′ terminal sequence distal to the label; and d) contacting the single-stranded overhang with an enzyme with 3′-5′ helicase activity under conditions wherein the first strand of the labeled polynucleotide is separated from the second strand up to the end proximal to the solid phase binding partner, at which point the desired polynucleotide is separated from the labeled polynucleotide.

In still another aspect, a method of preparing a desired polynucleotide comprising the steps of: a) preparing a labeled polynucleotide, wherein the labeled polynucleotide comprises a first strand having a 5′ terminus and a 3′ terminus, and wherein the labeled polynucleotide comprises target sequence between a double-stranded 5′ terminal sequence located proximal to the label, and a 3′ terminal sequence located distal to the label, the double-stranded 5′ terminal sequence including the 5′ terminus of the first strand and a sequence adjacent which includes one or more RNA bases, the 3′ terminal sequence including the 3′ terminus of the first strand and a sequence adjacent; b) immobilizing the labeled polynucleotide, said immobilizing comprising attaching the 5′ end of the double-stranded 5′ terminal sequence on the first strand to a solid phase binding partner; c) hybridizing a first target-specific primer to the target sequence; d) contacting the labeled polynucleotide and hybridized primer with an enzyme with polymerase activity under conditions wherein a second strand is synthesized, thereby creating a partially double-stranded fragment; e) introducing a nick or gap in the partially double-stranded fragment at the 5′ end of the double-stranded 5′ terminal sequence on the first strand proximal to the solid phase binding partner at the site of the one or more RNA bases; f) introducing one dideoxynucleotide in the nick or gap of step e), thereby creating a non-extendable break in the 5′ end of the double-stranded 5′ terminal sequence on the first strand proximal to the solid phase binding partner; g) removing any enzymes and unincorporated dideoxynucleotides from step f); h) hybridizing a second primer to a sequence upstream of the first target-specific primer, said second primer selected fro the group consisting of: (i) a second target-specific primer that hybridizes to a sequence on the target sequence; and (ii) a universal primer that hybridizes to a sequence on the 3′ terminal sequence; and g) contacting the labeled polynucleotide and hybridized second primer with an enzyme with exonuclease and polymerase activity or, in the alternative, with an enzyme with polymerase-strand-displacement activity, under conditions wherein a new second strand is synthesized up to a non-extendable location, at which point the desired polynucleotide is separated from the labeled polynucleotide.

In yet another aspect, a method of preparing a desired polynucleotide comprising the steps of: a) preparing a labeled polynucleotide, wherein the labeled polynucleotide comprises a first strand having a 5′ terminus and a 3′ terminus, and wherein the labeled polynucleotide comprises target sequence between a double-stranded 5′ terminal sequence located proximal to the label, and a 3′ terminal sequence located distal to the label, the double-stranded 5′ terminal sequence including the 5′ terminus of the first strand that contains 5 or more RNA bases, the 3′ terminal sequence including the 3′ terminus of the first strand and a sequence adjacent; b) hybridizing a first target-specific primer to the target sequence; c) contacting the labeled polynucleotide and hybridized primer with an enzyme with polymerase activity under conditions wherein a second strand is synthesized, thereby creating a partially double-stranded fragment; d) removing any enzymes and unincorporated deoxynucleotides from step c); e) introducing a break at the 5′ end of the double-stranded 5′ terminal sequence on the first strand proximal to the solid phase binding partner by removing the 5 or more RNA bases; f) hybridizing labeled probe in the break introduced in step e), wherein said probe is blocked from extension on the 3′ end and is attached to a solid support, thereby immobilizing the labeled polynucleotide; g) hybridizing a second primer to a sequence upstream of the first target-specific primer, said second primer selected fro the group consisting of: (i) a second target-specific primer that hybridizes to a sequence on the target sequence; and (ii) a universal primer that hybridizes to a sequence on the 3′ terminal sequence; and h) contacting the labeled polynucleotide and hybridized second primer with an enzyme with exonuclease and polymerase activity under conditions wherein a new second strand is synthesized up to a non-extendable location, at which point the desired polynucleotide is separated from the labeled polynucleotide.

In yet another aspect, a method of preparing a desired polynucleotide comprising the steps of: a) preparing a polynucleotide, wherein the polynucleotide comprises a first strand having a 5′ terminus and a 3′ terminus; b) hybridizing a labeled probe to the 5′ end of the first strand, wherein said probe is attached to a solid support, thereby immobilizing the labeled polynucleotide; c) hybridizing a target-specific primer to a sequence on the first strand that is 3′ to the hybridized probe; and d) contacting the labeled polynucleotide and hybridized primer with an enzyme with polymerase activity under conditions wherein a second strand is synthesized, thereby creating a partially double-stranded fragment that is separated from the probe.

In another aspect, a method of preparing a desired polynucleotide comprising the steps of: a) preparing a polynucleotide, wherein the polynucleotide comprises a first strand having a 5′ terminus and a 3′ terminus; b) hybridizing a target-specific, labeled probe to a sequence on the first strand, wherein said probe is attached to a solid support, thereby immobilizing the labeled polynucleotide; c) hybridizing a target-specific primer to a sequence on the first strand that is 3′ to the hybridized probe; and d) contacting the labeled polynucleotide and hybridized primer with an enzyme with polymerase activity under conditions wherein a second strand is synthesized, thereby creating a partially double-stranded fragment that is separated from the probe.

Other features and advantages of the present disclosure will become apparent from the following detailed description. It should be understood, however, that the detailed description and the specific examples, while indicating preferred embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 shows SPEED libraries and all elements of adaptor sequences “A” and “B” that are important for DNA size fractionation by SPEED method.

FIG. 2 shows one method to create a gap with a non-hydroxyl 3′ end by incubating a labeled polynucleotide comprising one or more dU residues with uracil glycosylase (UDG) and abasic endonuclease (such as endonuclease III or endonuclease VIII).

FIG. 3 shows one method to create an extendable nick or gap at the 3′ terminus distal to the label (and, thus solid support) by incubating a labeled polynucleotide comprising one or more RNA residues at the 3′ terminus (e.g., near the 5′ termini of the 3′-5′ second strand) with RNase H.

FIG. 4 shows one method to create an extendable nick or gap at the 3′ terminus distal to the label (and, thus solid support) by hybridizing and extending a first primer A1 to a labeled single-stranded polynucleotide, and then hybridizing a second primer A2 to a labeled single-stranded polynucleotide at a location upstream of the first primer A1.

FIG. 5 shows one method to create an extendable nick or gap at the distal 3′ terminus as well as a non-extendable nick or gap at the proximal 5′ terminus in one reaction by co-incubating a labeled polynucleotide comprising one or more dU residues at the 5′ terminus and further comprising one or more RNA residues at the 3′ terminus with uracil dU glycosylase (UDG), abasic endonuclease (such as endonuclease III or endonuclease VIII) and RNase H.

FIG. 6 shows one method to create an extendable nick or gap as well as a non-extendable nick or gap at both the 5′ and/or 3′ terminus regions by incubating a labeled polynucleotide comprising one or more RNA residues (e.g., at the 5′ terminus, shown in red color) and further comprising one or more RNA residues (e.g., at the 3′ terminus, shown in red color) with RNase H, DNA polymerase (e.g., Sequenase) and a specific dideoxynucleotide that become incorporated at the 5′ terminus but not at the 3′ terminus.

FIG. 7 shows mono-directional nick-translation based detachment of immobilized DNA.

FIG. 8 shows bi-directional nick-translation based detachment of immobilized DNA.

FIG. 9 shows mono-directional strand displacement based detachment of immobilized DNA.

FIG. 10 bi-directional strand displacement based detachment of immobilized DNA.

FIG. 11 shows mono-directional 5′-exonuclease based detachment of immobilized DNA.

FIG. 12 shows bi-directional 5′-exonuclease based detachment of immobilized DNA.

FIG. 13 shows mono-directional 3′-exonuclease based detachment of immobilized DNA.

FIG. 14 shows bi-directional 3′-exonuclease based detachment of immobilized DNA.

FIG. 15 shows mono-directional 5′-3′ helicase based detachment of immobilized DNA.

FIG. 16 shows mono-directional 3′-5′ helicase based detachment of immobilized DNA.

FIG. 17 shows mono-directional nick-translation based DNA size fractionation when immobilization is after DNA polymerization.

FIG. 18 shows the relationship between time and size of detached fragments.

FIG. 19 shows a process of DNA size-fractionation using the immobilized DNA library and the nick-translation reaction.

FIG. 20 shows TED-SPEED DNA sequence enrichment and depletion.

FIG. 21 shows TED-SPEED DNA sequence enrichment and depletion when immobilization is after first primer extension reaction and incubation with RNase H.

FIG. 22 shows the multiplexed TED-PLEX-SPEED method.

FIG. 23 shows a set of DNA fragments isolated by SPEED sequence selection method using target-specific primers Pa and Pb and a library of randomly fragmented DNA fragments.

FIG. 24 shows a set of DNA fragments isolated by SPEED sequence selection method using target-specific primer Pa, universal primer B (see FIGS. 20 and 21) and a library of randomly fragmented DNA fragments.

FIG. 25 shows a contigious DNA region isolated by SPEED sequence selection method using three pairs of primers and a library of randomly fragmented DNA fragments.

FIG. 26 shows a comparison of TED-SPEED-mediated PCR and conventional PCR as tools for DNA generation for sequencing by NGS methods.

FIG. 27 shows targeted nucleic acid enrichment by hybridization capture-polymerization detachment.

FIG. 28 shows the collection of a supernatant sample containing desired polynucleotides.

FIG. 29 shows an example of a DNA size sorter that is used with the materials and methods provided herein.

FIG. 30 shows an example of a DNA size analyzer that is used with the materials and methods provided herein.

FIG. 31 shows that target sequence(s) (single or multiplexed) can be detached and amplified individually.

FIG. 32 shows that target sequence(s) (single or multiplexed) are detached individually, but can be pooled at the end and amplified in one multiplexed PCR reaction.

FIG. 33 shows the genotyping method based on SPEED-mediated isolation and genome-wide sequence analysis of DNA regions containing CA-repeats.

FIG. 34 shows the enrichment of mutant alleles can substantially improve detection of mutant alleles and allow multiplex analyses of larger numbers of samples.

FIG. 35 shows a protocol for the targeted depletion of the wild-type K-ras codon.

FIG. 36A shows an electrophoregram of released DNA fragments, and

FIG. 36B shows gel electrophoresis of released DNA fragments using SPEED Size Selection Protocol.

FIG. 37 shows gel electrophoresis of DNA fragments released by SPEED Sequence Selection protocol.

### DESCRIPTION OF THE INVENTION

Unlike the known methods currently used to fractionate DNA that use chromatography principles, the methods provided herein do not involve gel or other chromatography media, but use enzymatic reactions to achieve the same goal. Compared to the known methods, the methods described herein offer many advantages that include, but are not limited to, simplicity (e.g., specificity, small amount of input DNA, use of common enzymatic reactions, and short run-times), high throughput capability, being amenable to automation, and performance in specialized devices, such as a DNA size sorter, or a DNA sequence sorter.

In one embodiment, the present methods (e.g., “SPEED-based size fractionation” or “Size Fractionation SPEED” or “SF-SPEED”) provide gel-free size fractionation processes. In another aspect, microarray-free targeted enrichment and amplification of nucleic acid sequences processes are provided. There are many situations when DNA or RNA need to be fractionated by size for downstream applications. Those situations include but are not limited to, cloning of PCR products, selection of a specific size fraction of genomic DNA digested by restriction endonuclease to reduce complexity of the DNA sample, micro-RNA isolation, preparation of genomic DNA and RNA libraries for de novo sequencing and targeted re-sequencing applications, and expression analysis using next-generation sequencing (NGS) platforms.

In another embodiment, the present methods provide sequence-specific fractionation processes. Unlike the known methods currently used to enrich DNA that use capture microarrays and targeted PCR amplification, the methods provided herein do not involve microarrays and conventional PCR, but use enzymatic reactions to achieve the same goals and do not require amplification. Advantages of the present methods (e.g., “DNA targeted enrichment/depletion by solid phase entrapment and enzymatic detachment” or DNA TED-SPEED” or “multiplexed targeted enrichment/depletion by SPEED method” or “TED-PLEX-SPEED”) include, but are not limited to, a universal library format (similar to capture-based methods), short protocol time (similar to PCR-based methods), reduced sequence bias, amplicons prepared in the NGS library format, useful for enrichment and depletion applications, amenable to automation, and they can be used to enrich mutated (rare) alleles. TED-SPEED and TED-PLEX-SPEED methods are useful, for example, in targeted enrichment of large DNA regions for re-sequencing by NGS methods, enrichment of regions containing micro-satellite repeats for genotyping by NGS, enrichment of regions containing specific promoter, enhancer, or gene family DNA for analysis by NGS, real-time PCR or other genotyping methods, depletion of wild alleles and enrichment of mutated alleles from DNA samples from cancer tissues for analysis by NGS, isolation and NGS analysis of transgenic elements with surrounding genomic DNA to establish the location of a transgene within the genome, and sequence analysis of novel microorganisms within complex bacterial pools by subtraction of the dominant bacterial DNA. Of course, the worker of ordinary skill in the art will readily appreciate that any number of molecular biological techniques and methods may be practiced in conjunction with the materials and methods provided herein.

### I. DEFINITIONS

As used herein, the term “polynucleotide” is used interchangeably with the term oligonucleotide. The term “nucleotide” or its plural as used herein is interchangeable with modified forms as discussed herein and otherwise known in the art. In certain instances, the art uses the term “nucleobase” which embraces naturally-occurring nucleotides as well as modifications of nucleotides that can be polymerized.

As used herein, the terms “desired polynucleotide,” “polynucleotide of a desired size,” or “polynucleotide of desired sequence” mean, in one aspect, a single-stranded polynucleotide. In another aspect the desired polynucleotide is double-stranded. In still another aspect the desired polynucleotide is a partially single-stranded and partially double-stranded. A desired polynucleotide includes, without limitation, a synthetic polynucleotide, a naturally-occurring polynucleotide, a chimeric polynucleotide that is a combination of a naturally-occurring polynucleotide and a synthetic polynucleotide, double-stranded-RNA, chromosomal DNA, plasmid DNA, viral DNA, mitochondrial DNA, phage DNA, bacterial DNA, or fragment thereof, sought to be selected by the practitioner of the disclosed methods.

As used herein, the term “labeled polynucleotide” or “label” refers to a moiety covalently attached to a polynucleotide. A labeled polynucleotide can comprise modification at the 5′-terminus, 3′terminus, a nucleobase, an internucleotide linkage, a sugar, amino, sulfide, hydroxyl, or carboxyl. Similarly, other modifications can be made at the indicated sites as deemed appropriate. As described herein, the methods of the present invention use labels as a means to immobilize polynucleotides. Thus, in various aspects, labels will have cognate binding partners that may be attached to a solid support. By way of example, biotin is a label according to the invention. Streptavidin and avidin and their derivatives are also labels according to the invention. Other labels can be represented by different haptens which can bind to corresponding antibodies immobilized to a solid support. By way of example, digoxigenin is a label according to the invention.

A “probe” or “labeled probe” or “capture probe” as used herein refers to a labeled polynucleotide useful for hybridizing to single-stranded polynucleotides or a single-stranded region within a partially single-stranded polynucleotide, in order to generate a labeled polynucleotide as described above.

As used herein, the term “immobilizing” means attaching to a solid support. As used herein, the term “support” or “solid support” is defined as a material having a rigid or semi-rigid surface. Such materials will preferably take the form of plates or slides, pellets, spherical beads, disks, capillary tubes or other convenient forms, although other forms may be used. In some embodiments, the beads are magnetic, while in other embodiments the beads are dielectric. In some embodiments, at least one surface of the solid support will be substantially flat. The solid support in various aspects is biological, non-biological, organic, inorganic, or a combination of any of these, existing as particles, strands, precipitates, gels, sheets, tubing, spheres, containers, capillaries, pads, slices, films, plates, slides, etc. The solid support is thus, in one aspect, flat but in other aspects, the support has alternative surface configurations. For example, in certain aspects, the solid support has raised or depressed regions on which reactions including, but not limited to, hybridization, ligation, and cleavage take place. In some embodiments, the solid support is chosen to provide appropriate light-absorbing characteristics. For example, the support in some aspects is a polymerized Langmuir Blodgett film, functionalized glass, Si, Ge, GaAs, GaP, SiO2, SiN4, modified silicon, or any one of a variety of gels or polymers such as (poly)tetrafluoroethylene, (poly)vinyliden-difluoride, polystyrene, polycarbonate, or combinations thereof. Other suitable solid support materials will be readily apparent to those of skill in the art. In certain aspects, the surface of the solid support includes reactive groups, which in various aspects are carboxyl, amino, hydroxyl, or thiol. In other aspects, the surface is optically transparent and in another aspect, the surface has Si—H functionalities, such as are found on silica surfaces. The solid support comprises, in various aspects, an array of ordered sets of dsDNA and/or ssDNA fragments that are covalently or non-covalently attached to the solid support.

As used herein, the term “5′ terminus” and “3′ terminus” refers to the 5′ and 3′ ends, or termini, of a polynucleotide respectively. The terms “5′ terminal sequence” and “3′ terminal sequence” refers to polynucleotide sequences at or near the termini, typically including “adaptor” sequences. “Adaptors” or “adaptor sequences” may be attached to the termini of polynucleotides to facilitate labeling according to the methods provided herein.

As used herein, the term “exonuclease activity” refers to an enzymatic activity involving cleavage of nucleotides one at a time from the end of a double-stranded polynucleotide chain. Such nucleotide cleavage constitutes a hydrolyzing reaction that breaks phosphodiester bonds at either the 3′ or the 5′ end. Exemplary 5′ exonucleases specific for double-stranded polynucleotides include, but are not limited to λ exonuclease and T7 gene 6 exonuclease. Exemplary 3′ exonucleases specific for double-stranded polynucleotides include, but are not limited to exonuclease III and DNA polymerases with 3′-5′ “proofreading” exonuclease activity such as DNA polymerase I, Klenow fragment, T4 DNA polymerase, T7 DNA polymerase, Phi 29 DNA polymerase, Pfu DNA polymerase, Vent DNA polymerase, Deep Vent DNA polymerase, Phusion DNA polymerase, etc. in the absence of dNTPs. As used herein, the term “nuclease-resistant base” refers to a base that is resistant to phosphodiester bond cleavage due to modifications in the linkage or base. Exemplary nuclease-resistant bases include, but are not limited to, nucleotides connected by modified linkages such as phosphorothioate and boranophosphate linkages, or nucleic acid analogs containing a 2′-O, 4′-C methylene bridge, also known as Locked Nucleic Acids, or LNAs. The term nuclease-resistant base refers also to a base that is resistant to exonuclease cleavage because of its structural location. Exemplary nuclease-resistant bases of this type of include 3′ single-stranded protruding bases in the case of the 3′ exonuclease III.

The term “nickase” or “nicking endonuclease” is understood in the art to mean an enzyme that recognizes a double-stranded polynucleotide and cleaves only one phosphodiester bond on one strand of a double-stranded polynucleotide. Thus, the term “nicking” means cleaving only one phosphodiester bond on one strand of a double-stranded polynucleotide. Exemplary commercially available nicking endonucleases include, but are not limited to, Nb.BbvCI, Nb.BsmI, Nb.BsrDI, Nb.BtsI, Nt.AlwI, Nt.BbvCI, Nt.BsmAI, Nt.BspQI, Nt.BstNBI and Nt.CviPII. All nickases introduce extendable nicks with 3′ hydroxyl ends.

As described herein, a nick or gap can also be introduced, for example, by a combination of uracil-dU-glycosylase (UDG) and abasic (i.e., AP or Apurinic/apyrimidinic) endonuclease. Depending on the type of AP endonuclease, the 3′ end may or may not have a 3′ hydroxyl group (resulting in either an extendable or non-extendable end). For example, extendable nicks or gaps may be introduced in a single step by RNase H or RNase H II enzymes if one of the duplex strands contains a stretch of 4 or more RNA bases (RNase H) or a single RNA base (RNase H II). Finally, a nick or gap can be created in two steps: step 1—degradation of the terminal DNA region by 5′ or 3′ exonuclease or by a combination of UDG/AP endonuclease if the 5′ (3′) terminal region contains multiple dU bases (incorporated during oligonucleotide synthesis); and step 2—hybridization of an oligonucleotide (with a 3′ hydroxyl, 3′ blocking group, or 3′ biotin group) complementary to the 3′ (5′) overhang resulting from step 1.

As used herein, the term “nick” or “gap” is defined as a region of a substantially double-stranded polynucleotide lacking integrity in one of the polynucleotide strands and missing no (e.g., a nick), one, or more consecutive bases (e.g., a gap) on one strand. According to the various embodiments of the disclosure, the gap is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, about 65, about 70, about 75, about 80, about 85, about 90, about 95, about 100, about 110, about 120, about 130, about 140, about 150 or more bases in length. As used herein, such nicks or gaps may be located in an interior region of polynucleotide or at the termini of a polynucleotide, including overhangs.

As used herein, the term “degraded” generally refers to the cleavage of phosphodiester bonds in a single-stranded or double-stranded polynucleotide such that the length of the polynucleotide chain is shortened incrementally over time.

As used herein, the term “non-degradable” refers to a location in a polynucleotide that cannot be degraded. By way of example, such a location in a polynucleotide contains one or more nuclease-resistant bases. The term “non-extendable,” as it relates to a location in a polynucleotide where polymerization stops and the desired polynucleotide separates from the label, refers to a location in a polynucleotide that cannot be extended. By way of example, a “non-extendable” 3′ end of a polynucleotide may contain such blocking groups as a dideoxynucleotide or a 3′-phosphate group. Similarly, the term “non-extendable” as it relates to a nick or gap with a blocked 3′ end, refers to a blocking group at the 3′ end of nick or gap) that blocks polymerase activity by precluding nick-translation or strand-displacement DNA synthesis from the 3′ terminus of the nick or gap. When present, the blocking group in certain aspects is a 3′ amino group, a 3′ phosphate, a dideoxynucleotide, a six carbon glycol spacer (e.g., hexanediol), or an inverted deoxythymidine (inverted dT). For example, inasmuch as a 3′ hydroxyl group is necessary for polymerase activity, the worker of ordinary skill will appreciate that any group other than a 3′ hydroxyl at the 3′ terminus of a nick or gap will be a useful blocking group.

“Enzyme collision location” as used herein refers to a location on a polynucleotide where two enzymes come into contact with one another. By way of example, both strands of a double-stranded polynucleotide with unprotected 5′-termini that are contacted by a 5′-3′ exonuclease would be degraded until the exonucleases reached the collision location, at which point the exonucleases may lose contact with its substrate strand. One of skill in the art would also readily appreciate that a non-extendable location as described above relates to an enzyme collision location inasmuch as collision of an enzyme (DNA polymerase, an exonuclease or a helicase) with a corresponding enzyme coming from the opposite direction would prohibit extension as a break in the template DNA strand.

The term “polymerase” is understood in the art to mean an enzyme or other catalyst capable of catalyzing a reaction leading to a template-sequence-dependent incorporation of a nucleotide at a 3′ end of a polynucleotide when the polynucleotide is annealed a complementary polynucleotide. Exemplary polymerases include but are not limited to Pfu DNA polymerase, Vent DNA polymerase, Vent (exo-) DNA polymerase, Deep Vent DNA polymerase, Deep Vent (exo-) DNA polymerase, E. coli polymerase I, T7 polymerase, reverse transcriptase, Taq DNA polymerase, DyNAzyme™ Ext DNA polymerase, Sequenase™, Klenow fragment, Bst polymerase large fragment, and the like.

As used herein, the term “fractionating” refers to separating molecules, e.g., polynucleotides, based on size (e.g., length) or sequence.

### II. POLYNUCLEOTIDES AND PREPARATION OF DNA LIBRARIES

### Polynucleotides

In various aspects, methods provided include use of polynucleotides which are DNA, modified DNA, RNA, modified RNA or combinations of the two types. Modified forms of polynucleotides are also contemplated for devices of the invention which include those having at least one modified internucleotide linkage. Modified polynucleotides or oligonucleotides are described in detail herein below.

In various aspects, the labeled or desired polynucleotide is about 100, about 120, about 140, about 160, about 180, about 200, about 250, about 300, about 350, about 400, about 450, about 500, about 600, about 700, about 800, about 900, about 1000, about 1100, about, about 1200, about 1300, about 1400, about 1500, about 1600, about 1700, about 1800, about 1900, about 2000, about 2500, about 3000, about 3500, about 4000, about 4500, about 5000, about 6000, about 7000, about 8000, about 9000, about 10,000, about 15,000, about 20,000, about 30,000, about 40,000, about 50,000, about 60,000, about 70,000, about 80,000, about 90,000, about 100,000, about 150,000, about 200,000, about 250,000, about 300,000, about 350,000, about 400,000, about 450,000, about 500,000, about 750,000, about 1,000,000, about 1,250,000, about 1,500,000, about 1,750,000, about 2,000,000, about 2,500,000, about 3,000,000, about 4,000,000, about 5,000,000, about 6,000,000, about 7,000,000, about 8,000,000, about 9,000,000, about 10,000,000 or more bases in length. It will be understood that the term “about” as used herein means “approximately.”

In various aspects, a labeled or desired polynucleotide for use with the methods of the invention is a single stranded polynucleotide or a double-stranded polynucleotide. In another aspect, the labeled or desired polynucleotide is a substantially double-stranded polynucleotide molecule that has internal single-stranded regions with free 3′ ends. By “substantially double-stranded” it is meant that greater than about 75%, greater than about 80%, greater than about 85%, greater than about 90%, greater than about 95%, greater than about 98%, or greater than about 99% of the labeled or desired polynucleotide is double-stranded. In another aspect, the labeled or desired polynucleotide substantially double-stranded polynucleotide molecule is about 100, about 120, about 140, about 160, about 180, about 200, about 250, about 300, about 350, about 400, about 450, about 500, about 600, about 700, about 800, about 900, about 1000, about 1100, about, about 1200, about 1300, about 1400, about 1500, about 1600, about 1700, about 1800, about 1900, about 2000, about 2500, about 3000, about 3500, about 4000, about 4500, about 5000, about 6000, about 7000, about 8000, about 9000, about 10,000, about 15,000, about 20,000, about 30,000, about 40,000, about 50,000, about 60,000, about 70,000, about 80,000, about 90,000, about 100,000, about 150,000, about 200,000, about 250,000, about 300,000, about 350,000, about 400,000, about 450,000, about 500,000, about 750,000, about 1,000,000, about 1,250,000, about 1,500,000, about 1,750,000, about 2,000,000, about 2,500,000, about 3,000,000, about 4,000,000, about 5,000,000, about 6,000,000, about 7,000,000, about 8,000,000, about 9,000,000, about 10,000,000 or more bases in length.

Methods for polynucleotide hybridization and washing are well known in the art and can be found in standard references in molecular biology such as those cited herein. In general, hybridizations are usually carried out in solutions of high ionic strength (6×SSC or 6×SSPE) at a temperature 20° C. to 25° C. below the melting temperature (Tm). High stringency wash conditions are often determined empirically in preliminary experiments, but usually involve a combination of salt and temperature that is approximately 12° C. to 20° C. below the Tm. One example of high stringency wash conditions is 1×SSC at 60° C. Another example of high stringency wash conditions is 0.1×SSPE, 0.1% SDS at 42° C. (Meinkoth and Wahl, Anal. Biochem., 138:267-284, 1984). An example of even higher stringency wash conditions is 0.1×SSPE, 0.1% SDS at 50° C. to 65° C. In another non-limiting example, high stringency washing is carried out under conditions of 1×SSC and 60° C. As is well recognized in the art, various combinations of factors can result in conditions of substantially equivalent stringency. Such equivalent conditions are within the scope of the present disclosure. Formulas standard in the art are appropriate for determining exact hybridization conditions. See Sambrook et al., 9.47-9.51 in Molecular Cloning, Cold Spring Harbor Laboratory Press, Cold Spring Harbor, N.Y. (1989).

In various aspects, modified polynucleotides are provided by the invention. For example, modified polynucleotides may be used in the terminal or adaptor sequences of labeled polynucleotides as described herein. Specific examples of modified polynucleotides include those containing modified backbones or non-natural internucleoside linkages. Polynucleotides having modified backbones include those that retain a phosphorus atom in the backbone and those that do not have a phosphorus atom in the backbone. Modified polynucleotides that do not have a phosphorus atom in their internucleoside backbone are considered to be within the meaning of “polynucleotides.”

Modified polynucleotides backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are polynucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified polynucleotides backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH2 component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In still other embodiments, polynucleotides mimetics wherein both one or more sugar and/or one or more internucleotide linkage of the nucleotide units are replaced with “non-naturally occurring” groups. In one aspect, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., 1991, Science, 254: 1497-1500, the disclosures of which are herein incorporated by reference.

In still other embodiments, polynucleotides are provided with phosphorothioate backbones and oligonucleosides with heteroatom backbones, and including —CH2-NH—O—CH2-, —CH2-N(CH3)-O—CH2-, —CH2-O—N(CH3)-CH2-, —CH2-N(CH3)-N(CH3)-CH2- and —O—N(CH3)-CH2-CH2— described in U.S. Pat. Nos. 5,489,677, and 5,602,240. Also contemplated are polynucleotides with morpholino backbone structures described in U.S. Pat. No. 5,034,506.

In various forms, the linkage between two successive monomers in the oligo consists of 2 to 4, desirably 3, groups/atoms selected from —CH2-, —O—, —S—,—NRH—, >C═O, >C═NRH, >C═S, —Si(R″)2-, —SO—, —S(O)2-, —P(O)2-, PO(BH3)-, —P(O,S)—, —P(S)2-, —PO(R″)—, —PO(OCH3)-, and —PO(NHRH)—, where RH is selected from hydrogen and C1-4-alkyl, and R″ is selected from C1-6-alkyl and phenyl. Illustrative examples of such linkages are —CH2-CH2-CH2-, —CH2-CO—CH2-, —CH2-CHOH—CH2-, —O—CH2-O—, —O—CH2-CH2-, —O—CH2-CH═(including R5 when used as a linkage to a succeeding monomer), —CH2-CH2-O—, —NRH—CH2-CH2-, —CH2-CH2-NRH—, —CH2-NRH—CH2-, —O—CH2-CH2-NRH—, —NRH—CO—O—, —NRH—CO—NRH—, —NRH—CS—NRH—, NRH—C(═NRH)—NRH—, —NRH—CO—CH2-NRH—O—CO—O—, —O—CO—CH2-O—, —O—CH2-CO—O—, —CH2-CO—NRH—, —O—CO—NRH—, NRH—CO—CH2-, —O—CH2-CO—NRH—, —O—CH2-CH2-NRH—, —CH═N—O—, —CH2-NRH—O—, —CH2-O—N═(including R5 when used as a linkage to a succeeding monomer), —CH2-O—NRH—, —CO—NRH—CH2-, —CH2-NRH—O—, —CH2-NRH—CO—, —O—NRH—CH2-, —O—NRH, —O—CH2-S—, S—CH2-O—, —CH2-CH2-S—, —O—CH2-CH2-S—, —S—CH2-CH═(including R5 when used as a linkage to a succeeding monomer), —S—CH2-CH2-, —S—CH2-CH2-O—, —S—CH2-CH2-S—, —CH2-S—CH2-, —CH2-SO—CH2-, —CH2-SO2-CH2-, —O—SO—O—, —O—S(O)2-O—, —O—S(O)2-CH2-, —O—S(O)2-NRH—, —NRH—S(O)2-CH2-; —O—S(O)2-CH2-, —O—P(O)2-O—, —O—P(O,S)—O—, —O—P(S)2-O—, —S—P(O)2-O—, —S—P(O,S)—O—, —S—P(S)2-O—, —O—P(O)2-S—, —O—P(O,S)—S—, —O—P(S)2-S—, —S—P(O)2-S—, —S—P(O,S)—S—, —S—P(S)2-S—, —O—PO(R″)—O—, O—PO(OCH3)-O—, —O—PO(O CH2CH3)-O—, —O—PO(O CH2CH2S—R)—O—, —O—PO(BH3)-O—, —O—PO(NHRN)—O—, —O—P(O)2-NRH H—, —NRH—P(O)2-O—, —O—P(O,NRH)—O—, —CH2-P(O)2-O—, —O—P(O)2-CH2-, and —O—Si(R″)2-O—; among which —CH2-CO—NRH—, —CH2-NRH—O—, —S—CH2-O—, —O—P(O)2-O—O—P(—O,S)—O—, —O—P(S)2-O—, —NRH P(O)2-O—, —O—P(O,NRH)—O—, —O—PO(R″)—O—, —O—PO(CH3)-O—, and —O—PO(NHRN)—O—, where RH is selected form hydrogen and C1-4-alkyl, and R″ is selected from C1-6-alkyl and phenyl, are contemplated. Further illustrative examples are given in Mesmaeker et. al., 1995, Current Opinion in Structural Biology, 5: 343-355 and Susan M. Freier and Karl-Heinz Altmann, 1997, Nucleic Acids Research, vol 25: pp 4429-4443.

Still other modified forms of polynucleotides are described in detail in U.S. patent application NO. 20040219565, the disclosure of which is incorporated by reference herein in its entirety.

Modified polynucleotides also optionally contain one or more substituted sugar moieties. In certain aspects, oligonucleotides comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C1 to C10 alkyl or C2 to C10 alkenyl and alkynyl. Other embodiments include O[(CH2)nO]mCH3, O(CH2)nOCH3, O(CH2)nNH2, O(CH2)nCH3, O(CH2)nONH2, and O(CH2)nON[(CH2)nCH3]2, where n and m are from 1 to about 10. Other oligonucleotides comprise one of the following at the 2′ position: C1 to C10 lower alkyl, substituted lower alkyl, alkenyl, alkynyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In one aspect, a modification includes 2′-methoxyethoxy (2′—O—CH2CH2OCH3, also known as 2′—O-(2-methoxyethyl) or 2′-MOE) (Martin et al., 1995, Helv. Chim. Acta, 78: 486-504) i.e., an alkoxyalkoxy group. Other modifications include 2′-dimethylaminooxyethoxy, i.e., a O(CH2)2ON(CH3)2 group, also known as 2′-DMAOE, as described in examples herein below, and 2′-dimethylaminoethoxyethoxy (also known in the art as 2′—O-dimethyl-amino-ethoxy-ethyl or 2′-DMAEOE), i.e., 2′—O—CH2-O—CH2-N(CH3)2, also described in examples herein below.

Still other modifications include 2′-methoxy (2′—O—CH3), 2′-aminopropoxy (2′—OCH2CH2CH2NH2), 2′-allyl (2′-CH2-CH═CH2), 2′—O-allyl (2′—O—CH2-CH═CH2) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In one aspect, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects is a methylene (—CH2-)n group bridging the 2′ oxygen atom and the 4′ carbon atom wherein n is 1 or 2. LNAs and preparation thereof are described in WO 98/39352 and WO 99/14226.

Polynucleotides also include base modifications or substitutions. As used herein, “unmodified” or “natural” bases include the purine bases adenine (A) and guanine (G), and the pyrimidine bases thymine (T), cytosine (C) and uracil (U). Modified bases include other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′, 2′: 4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′—O-methoxyethyl sugar modifications. See, U.S. Pat. Nos. 3,687,808, U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

A “modified base” or other similar term refers to a composition which can pair with a natural base (e.g., adenine, guanine, cytosine, uracil, and/or thymine) and/or can pair with a non-naturally occurring base. In certain aspects, the modified base provides a Tm differential of 15, 12, 10, 8, 6, 4, or 2° C. or less. Exemplary modified bases are described in EP 1 072 679 and WO 97/12896.

By “nucleobase” is meant the naturally occurring nucleobases adenine (A), guanine (G), cytosine (C), thymine (T) and uracil (U) as well as non-naturally occurring nucleobases such as xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” thus includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). The term “nucleosidic base” or “base unit” is further intended to include compounds such as heterocyclic compounds that can serve like nucleobases including certain “universal bases” that are not nucleosidic bases in the most classical sense but serve as nucleosidic bases. Especially mentioned as universal bases are 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

### Preparation of DNA Libraries

Methods of making polynucleotides having a predetermined sequence are well-known in the art. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both oligoribonucleotides and oligodeoxyribonucleotides (the well-known methods of synthesizing DNA are also useful for synthesizing RNA). Oligoribonucleotides and oligodeoxyribonucleotides can also be prepared enzymatically.

The methods provided herein allow fractionation of polynucleotides of different origins. For example, DNA digested with restriction enzymes, DNA cleaved by non-specific nucleases, DNA fragmented by mechanical methods such as sonication, nebulization, hydro-shearing, DNA fragmented by chemical methods, DNA fragmented by heating, DNA amplified by PCR, DNA amplified by isothermal amplification methods, cDNA produced by reverse-transcription synthesis, synthetic DNA, as well as different types of RNA molecules such as total RNA, mRNA, microRNA, and the like.

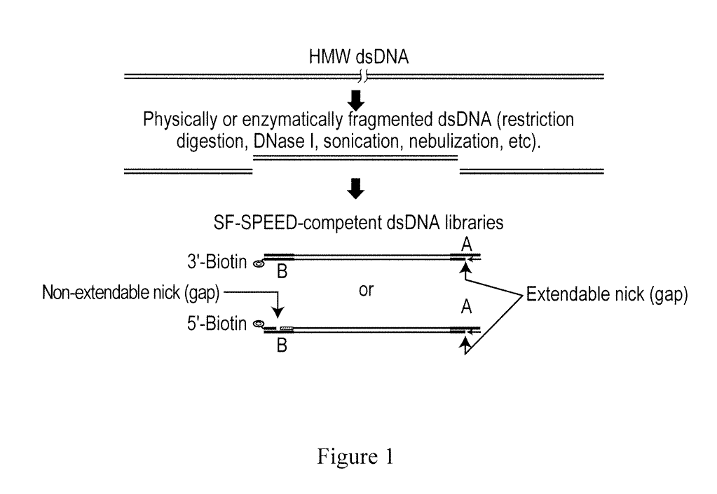

In various aspects of the invention, preparation of “in vitro” DNA libraries for DNA size fractionation using SF-SPEED (as opposed to “in vivo” DNA libraries used in bacterial DNA cloning) is provided. In this way, “adaptors” or “adaptor sequences” are attached to the termini of fragmented double-stranded DNA (dsDNA) as illustrated in FIG. 1. As shown in FIG. 1, the adaptor sequences are labeled “A” and “B.” One of skill in the art will appreciate that numerous methods exist that allow attachment of adaptor sequences to polynucleotides. Such methods include, but are not limited to, PCR, ligation-based methods, ligation-polymerization-based methods, polymerization-based methods, terminal transferase (TdT)-based methods, and transposon-based methods.

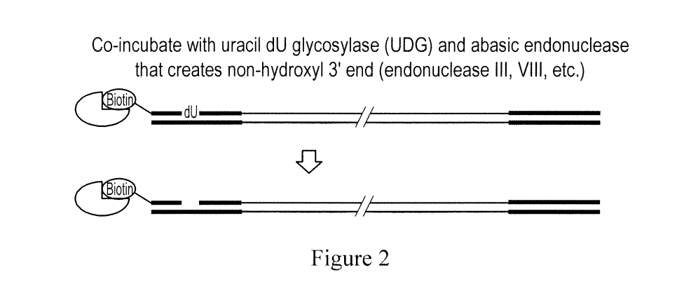

In various aspects, extendable and non-extendable nicks or gaps are created according to methods provided herein (e.g., in Sections III and IV, below). In one aspect, a non-extendable nick or gap may be created at the 5′ terminus proximal to the label (and, thus solid support) by incubating a labeled polynucleotide comprising one or more dU residues with uracil glycosylase (UDG) and abasic endonuclease to create a non-hydroxyl 3′ end; as shown in FIG. 2.

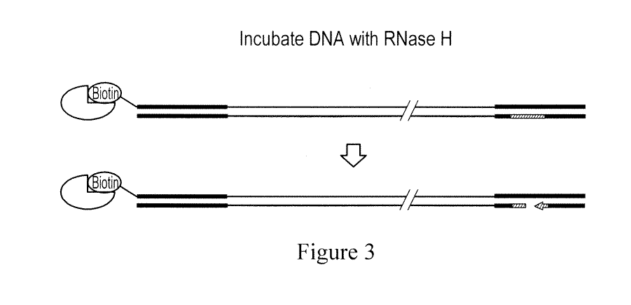

In another aspect, an extendable nick or gap may be created at the 3′ terminus distal to the label (and, thus solid support) by incubating a labeled polynucleotide comprising one or more RNA residues at the 3′ terminus (e.g., near the 5′ termini of the 3′-5′ second strand) with RNase H; as shown in FIG. 3.

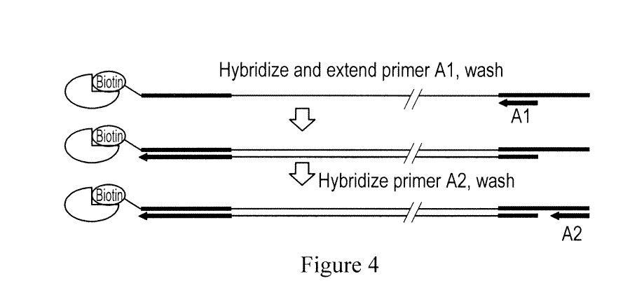

In another aspect, an extendable nick or gap may be created at the 3′ terminus distal to the label (and, thus solid support) by hybridizing and extending a first primer to a labeled single-stranded polynucleotide, and then hybridizing a second primer to a labeled single-stranded polynucleotide at a location upstream of the first primer, as shown in FIG. 4.

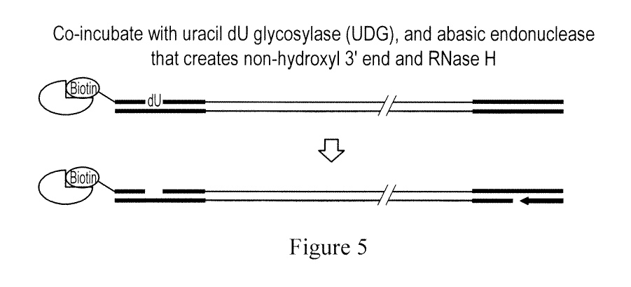

In another aspect, an extendable nick or gap at the distal 3′ terminus as well as a non-extendable nick or gap at the proximal 5′ terminus may be created in one reaction by co-incubating a labeled polynucleotide comprising one or more dU residues at the 5′ terminus and further comprising one or more RNA residues at the 3′ terminus with uracil dU glycosylase (UDG), abasic endonuclease and RNase H, as shown in FIG. 5.

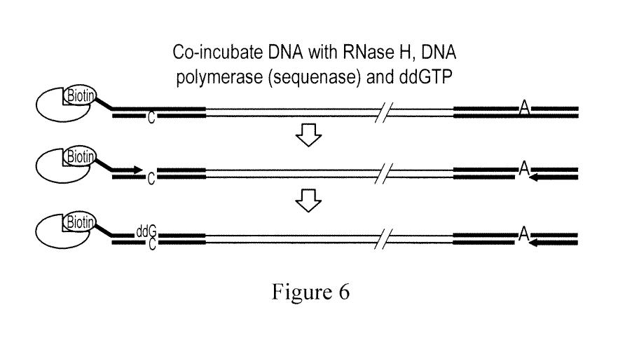

In another aspect, an extendable nick or gap as well as a non-extendable nick or gap may be created at both the 5′ and/or 3′ terminus regions by incubating a labeled polynucleotide comprising one or more RNA residues (e.g., at the 5′ terminus) and further comprising one or more RNA residues (e.g., at the 3′ terminus) with RNase H, DNA polymerase (e.g., Sequenase) and a specific dideoxynucleotide, as shown in FIG. 6. FIG. 6 shows an example that utilizes dideoxynucleotide ddGTP which is incorporated by a DNA polymerase into the proximal 5′ terminus (complementary cytosine in the downstream location of the template strand of the 5′ terminus) but cannot be incorporated into the distal 3′ terminus (non-complementary adenine in the downstream location of the template strand of the 3′ terminus).

### III. FRACTIONATION OF POLYNUCLEOTIDES BY SIZE

The methods provided herein require manipulation of various polynucleotide structures (e.g., single-stranded DNA (ssDNA), dsDNA, and the like) using various molecular biological techniques.

In various embodiments of the disclosure, a polynucleotide of a desired size is prepared by contacting a labeled polynucleotide having a double-stranded region comprising a first strand and a second strand with an enzyme (e.g., an enzyme capable of catalyzing nick-translation polymerization, an enzyme capable of catalyzing strand-displacement polymerization, an enzyme capable of catalyzing double-stranded polynucleotide-specific degradation, or an enzyme capable of catalyzing polynucleotide strand unwinding) or an enzyme mixture (e.g., a mixture of a 5′ exonuclease and DNA polymerase lacking 5′ nuclease activity to create “a nick-translation” activity), under conditions wherein the interaction of the first strand with the second strand is reduced, thereby resulting in dissociation of the second strand from the first strand. As used herein, “under conditions wherein the interaction of the first strand with the second strand is reduced, thereby resulting in dissociation of the second strand from the first strand,” means that the enzymatic activity results in a reduction in the interaction (e.g., hydrogen bonding) between the first and second strands to an extent such that the number of interaction sites (e.g., hydrogen bonds) is insufficient to maintain a stable interaction.

In some aspects of the disclosure, a labeled polynucleotide has at least one region that is single stranded. In instances wherein the target polynucleotide molecule is double-stranded, the single stranded region may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, about 65, about 70, about 75, about 80, about 85, about 90, about 95, about 100 or more bases in length.

In some aspects of the disclosure, a phosphodiester bond is cleaved on one strand of the labeled polynucleotide. In specific aspects, the phosphodiester bond is cleaved by a nicking endonuclease.

In various embodiments of the disclosure, a single stranded region is generated on the labeled polynucleotide by nick-mediated exonuclease DNA degradation, or nick-mediated strand-displacement DNA synthesis. Nick-mediated exonuclease DNA degradation is described by and illustrated below. Briefly, after a phosphodiester bond is cleaved by a nicking endonuclease, a labeled polynucleotide molecule is incubated with an exonuclease. In one aspect, the exonuclease is a 3′ exonuclease. In another aspect, the exonuclease is a 5′ exonuclease. In some aspects, the exonuclease is heat inactivated to stop exonuclease activity. The single stranded region generated by the exonuclease may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, about 65, about 70, about 75, about 80, about 85, about 90, about 95, about 100 or more bases in length. As will be understood by one of skill in the art, the desired length of the single stranded region can be regulated by varying the amount of time the target polynucleotide is exposed to the exonuclease.

### A. Polymerization-Mediated Size Fractionation

In one embodiment, the invention provides materials and methods for polymerization-mediated DNA size fractionation. In various aspects, the size fractionation is mono-directional or bi-directional. As described herein, polymerization-mediated size fractionation includes methods wherein a desired polynucleotide is detached from a labeled polynucleotide using nick-translation or strand displacement, as illustrated below, the following major steps generally occur in such methods although, as described herein, these steps need not necessarily occur in this order:

(1) Immobilization of double-stranded DNA fragments by either 3′ or 5′ end;

(2) Creation of a non-extendable break (e.g., gap) within the proximal (to a solid support) end of DNA fragments if the attachment involves the 5′ end by either a) cleaving with an enzyme or group of enzymes that create a 3′ non-hydroxyl end at the nick or gap, b) cleaving with an enzyme or a group of enzymes that create a 3′ hydroxyl end at the nick or gap and then blocking the 3′ hydroxyl end by introducing a dideoxynucleotide at the nick or gap, or c) by partially degrading the 5′ end of the terminal sequence on the 5′-3′ strand (e.g., first strand) proximal to the solid phase binding partner and hybridizing a 3′ end-blocked oligonucleotide with a label at the 5′ end, thereby creating a non-extendable break (e.g., gap) in the 5′ end of the terminal sequence on the 5′-3′ strand (e.g., first strand) proximal to the solid phase binding partner;

(3) Creation of an extendable break (e.g., gap) within the distal end of DNA fragments (e.g., by cleaving with an enzyme or a group of enzymes that create a 3′ hydroxyl end at the nick or gap, or 2) by partially degrading the 5′ end of the terminal sequence on the 3′-5′ strand distal to the solid phase binding partner and hybridizing an oligonucleotide with a hydroxyl group at the 3′ end); and

(4) Initiation of polymerization-mediated size fractionation at the distal end.

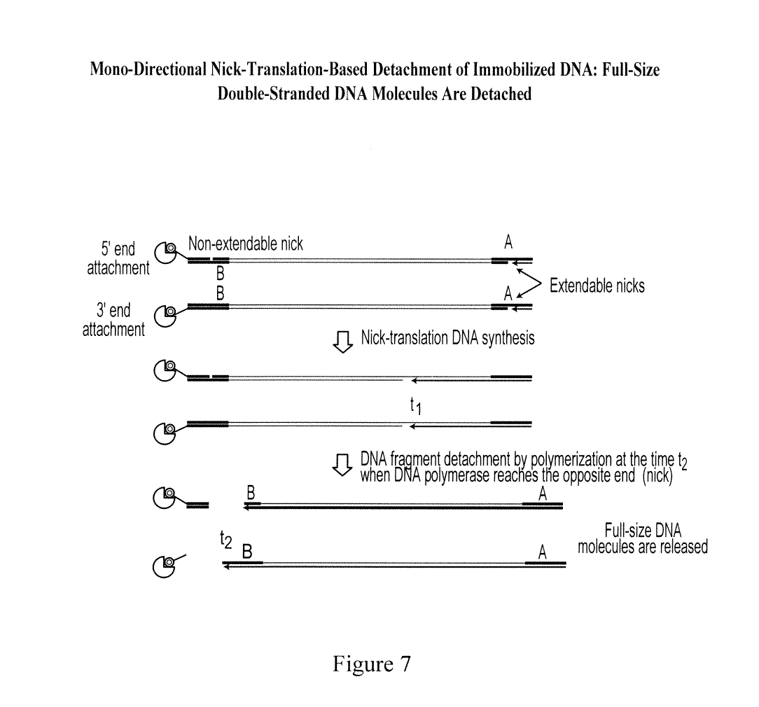

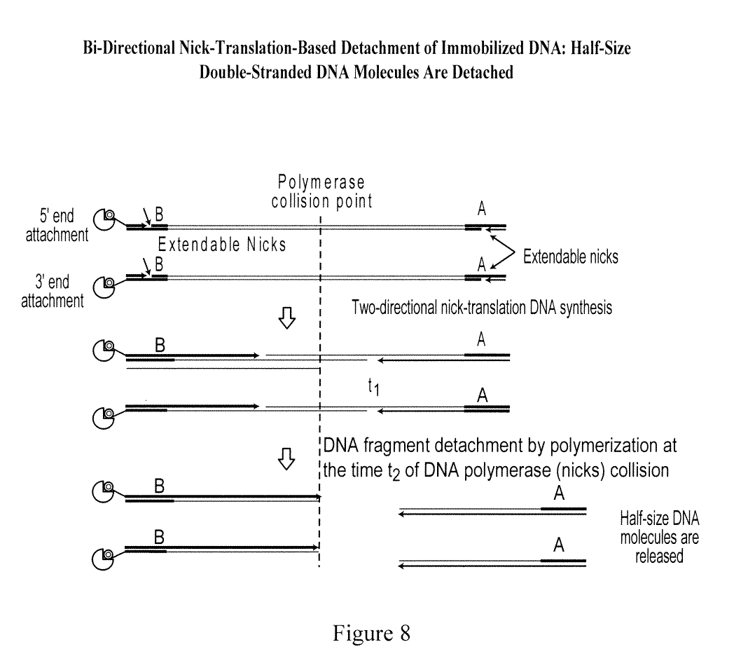

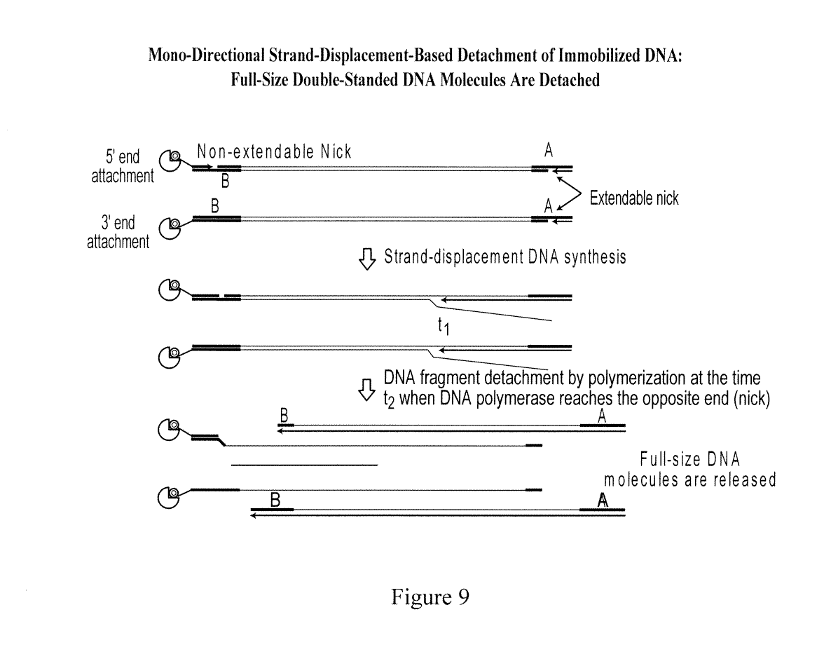

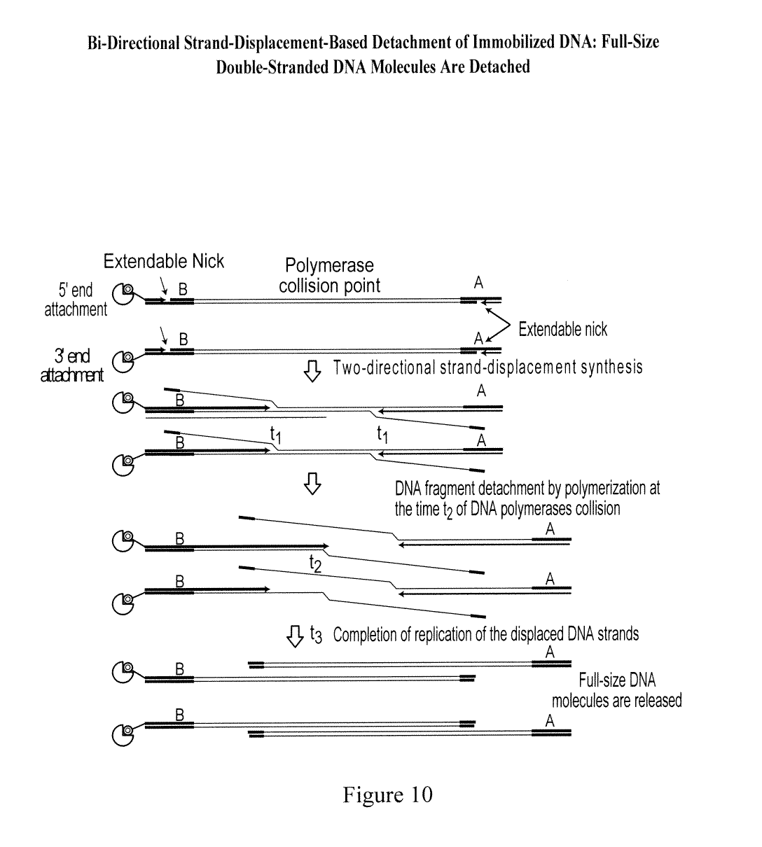

In this way, DNA fragments will be released in a sequential (by size) order as soon as the enzyme reaches the opposite DNA end (mono-directional) or when two enzymes collide in the middle of the DNA fragment (bi-directional). Steps (1)-(4) above relate to a mono-directional polymerization-mediated method which releases DNA molecules of the full size. Bi-directional methods are also provided by the invention and result in release of half-size molecules. Of course, one of skill in the art would appreciate that step (2) above would be replaced by step (3) for the bi-directional methods, thereby generating extendable break (e.g., gap)s at each termini. FIGS. 7, 8, 9 and 10 describe the nick-translation and strand displacement methods.

### B. Exonuclease-Mediated Size Fractionation

In another embodiment, the invention provides materials and methods for exonuclease-mediated DNA size fractionation. In various aspects, the size fractionation is mediated by a 5′ exonuclease or a 3′ exonuclease. In various aspects, the size fractionation is mono-directional or bi-directional. As illustrated below, the following major steps generally occur in such methods although, as described herein, these steps need not necessarily occur in this order:

(1) Immobilization of double-stranded DNA fragments by one of their 3′- or 5′-ends;

(2) In the case of immobilization through the 5′-end and use of the 5′ exonuclease, by for example, cleaving with an enzyme or group of enzymes that create a nick or gap within the 5′ end proximal to the solid support or, partially degrading the 5′ end of the terminal sequence on the 5′-3′ strand (e.g., first strand) proximal to the solid phase binding partner and hybridizing an oligonucleotide with a label, thereby creating a break (e.g., gap) in the 5′ end of the terminal sequence on the 5′-3′ strand (e.g., first strand) proximal to the solid phase binding partner;

(2A) In the case of immobilization through the 3′-end and use of the 3′ exonuclease, by for example, cleaving with an enzyme or group of enzymes that create a nick or gap within the 3′ end proximal to the solid support or, partially degrading the 3′ end of the terminal sequence on the 3′-5′ strand (e.g., second strand) proximal to the solid phase binding partner and hybridizing an oligonucleotide with a label, thereby creating a break (e.g., gap) in the 3′ end of the terminal sequence on the 3′-5′ strand (e.g., second strand) proximal to the solid phase binding partner;

(3) Optionally, protection of the 5′-termini proximal to the solid support (or 3′-termini in the case of 3′ immobilization) from 5′-exonuclease (or 3′ exonuclease) degradation by introducing nuclease-resistant bases or single-stranded 5′-flap (or 3′-flap);

(4) Initiation of 5′-exonuclease (or 3′-exonuclease)-mediated DNA degradation reaction. DNA fragments will be released in a sequential (by size) order as soon as the exonuclease(s) would reach the opposite DNA end (mono-directional degradation) or collide in the middle of a DNA fragment (bi-directional degradation).

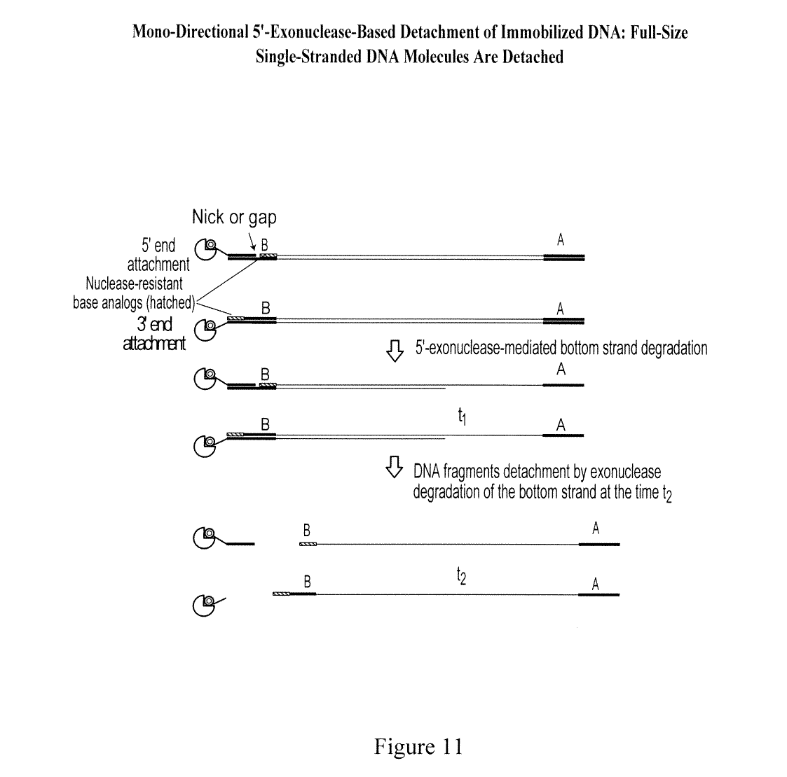

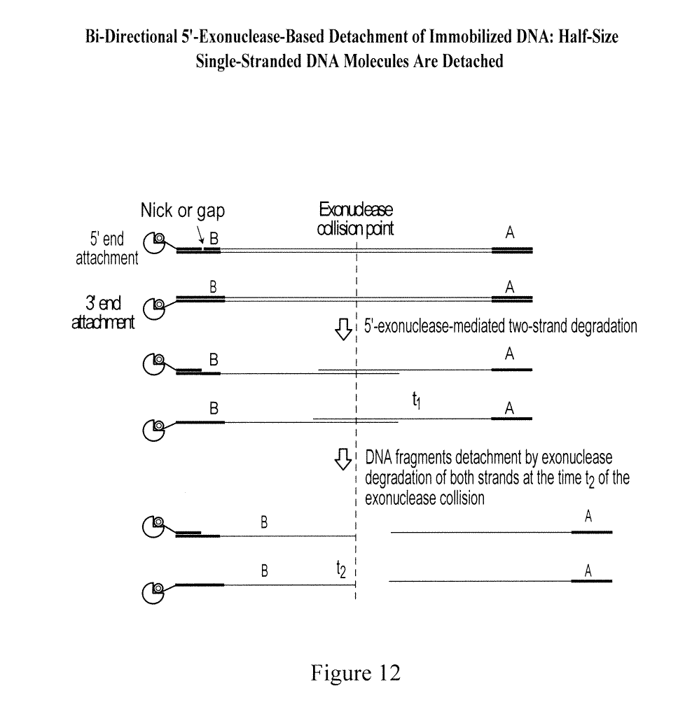

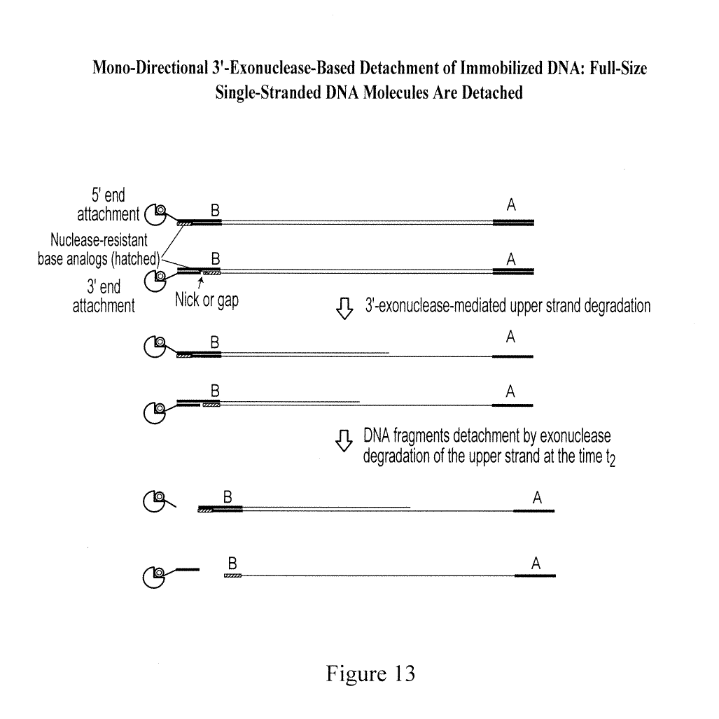

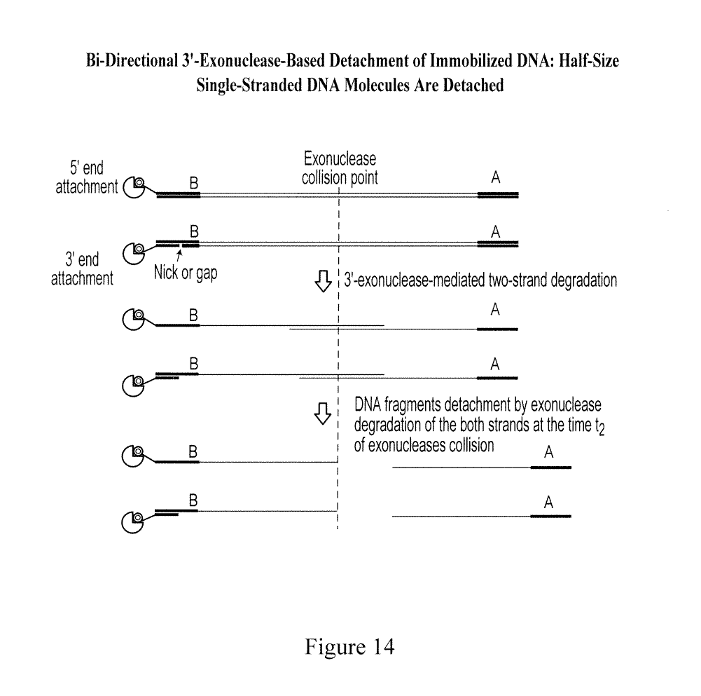

Of course, as illustrated in FIGS. 11, 12, 13 and 14 and as one of skill in the art would appreciate, for bi-directional methods step (3) above would be omitted. FIGS. 11, 12, 13 and 14 further describe exonuclease-mediated DNA size fractionation.

### C. Helicase-Mediated Size Fractionation

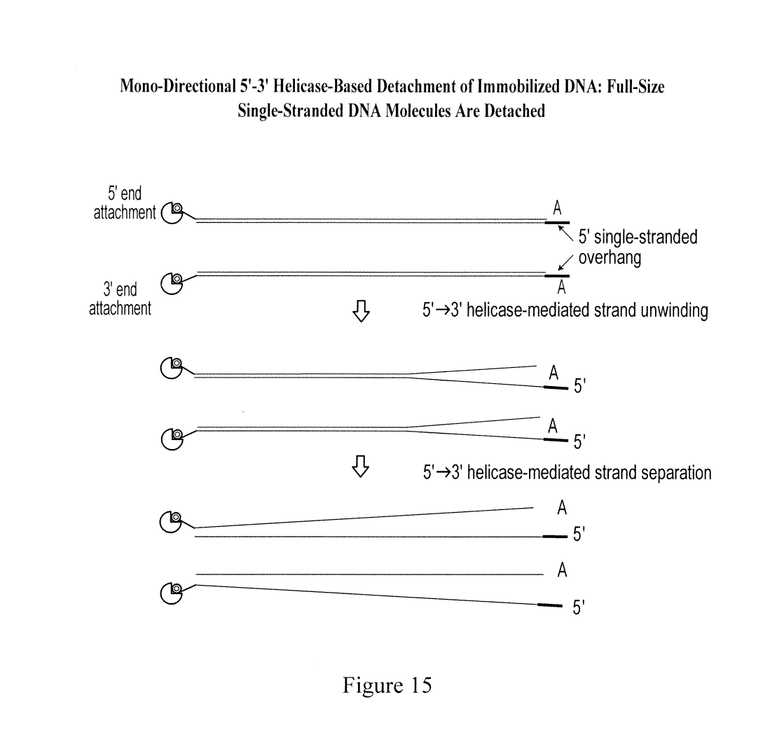

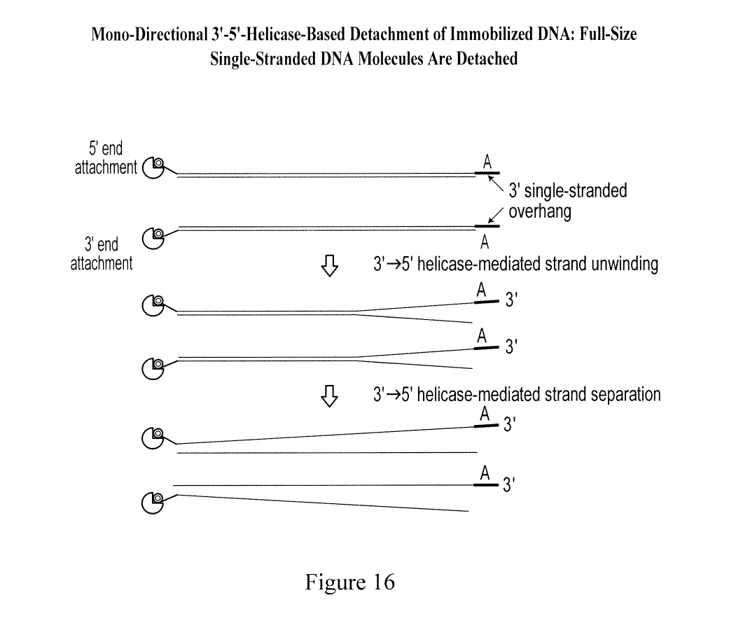

In another embodiment, the invention provides materials and methods for helicase-mediated DNA size fractionation. In various aspects, the size fractionation is mediated by a 5′-3′ helicase or a 3′-5′ helicase. As illustrated in FIGS. 15 and 16, the following major steps generally occur in such methods although, as described herein, these steps need not necessarily occur in this order: (1) Immobilization of double-stranded DNA fragments by 3′ or 5′ end of the DNA end proximal to the solid support; (2) Introduction of a 5′ or 3′ single-stranded tail at the distal end of the DNA fragment; and (3) Initiation of 5′-3′ or 3′-5′ helicase-mediated DNA unwinding reaction. DNA fragments will be released in a sequential (by size) order as soon as the helicase reaches the opposite DNA end. FIGS. 15 and 16 further describe helicase-mediated DNA size fractionation.

### D. Immobilization

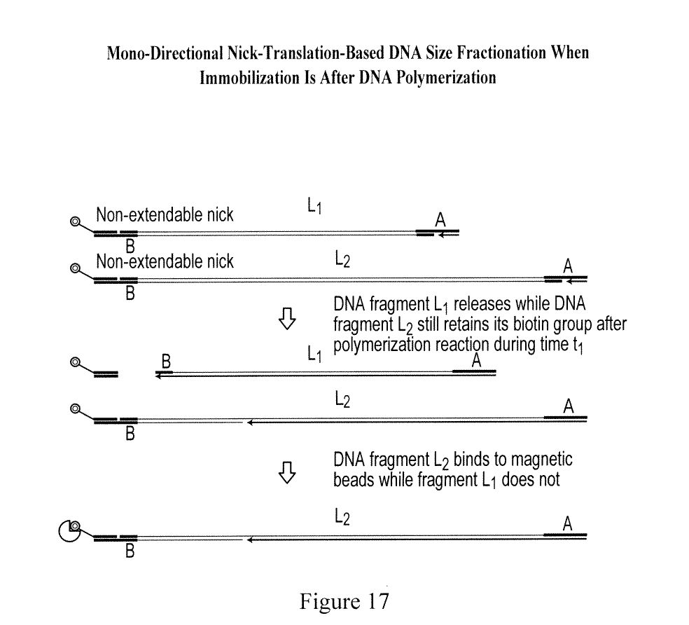

The immobilization of DNA to solid phase can be covalent, involve streptavidin-biotin or antibody-hapten interaction, or, in some cases, hybridization. The immobilization step of the methods provided herein need not necessarily occur prior to, e.g., contacting the labeled polynucleotide with an enzyme (e.g., nuclease, polymerase, helicase, etc.). For example, as shown in FIG. 17, immobilization may occur after the labeled polynucleotide is contacted with, e.g., a polymerase:

### E. Reaction Times

Four types of enzymatic reactions that include polymerase-mediated nick-translation, strand-displacement polymerization reaction, 5′ or 3′ exonuclease-mediated DNA degradation, and helicase-mediated DNA unwinding reactions are used in various embodiments to release immobilized DNA. It is assumed that the synthesis or the nuclease degradation rate, or unwinding rate is sequence-independent and easily controlled by time.

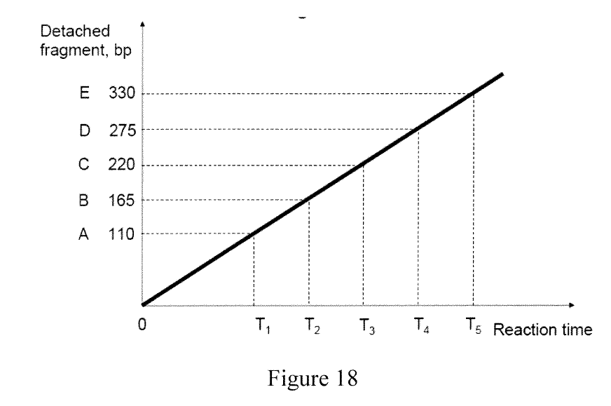

A linear relationship exists between polymerization (nick-translation) reaction time and the size of detached fragments, as illustrated in FIG. 18.

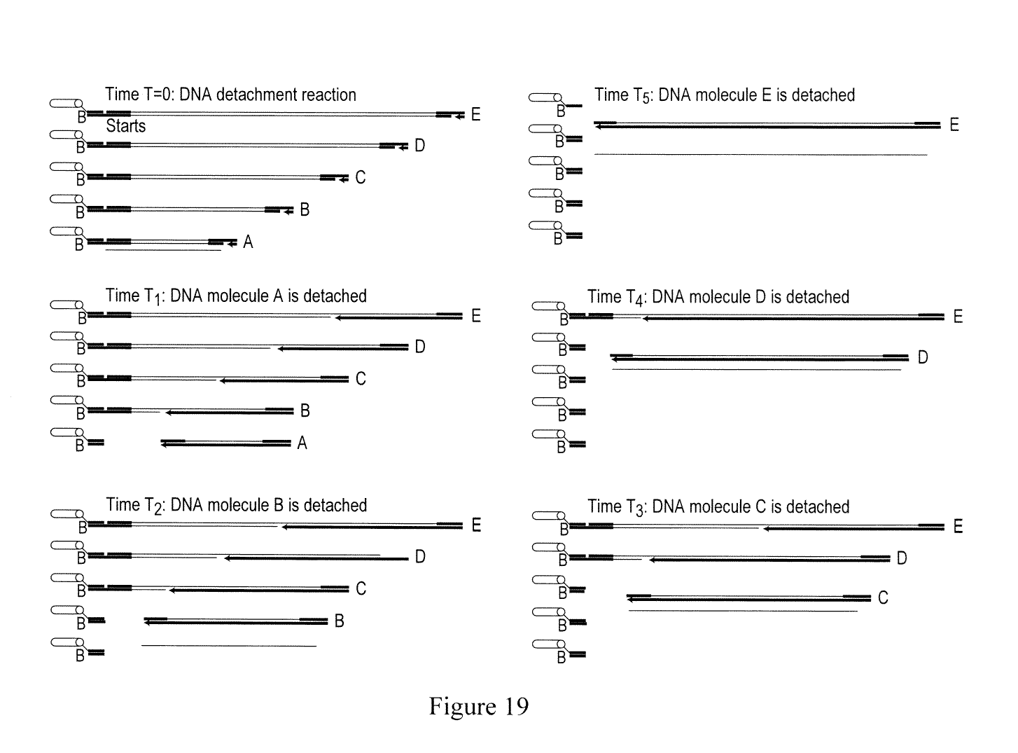

A process of DNA size-fractionation using the immobilized DNA library and the nick-translation reaction is illustrated in FIG. 19 showing step-by-step release of DNA fragments A, B, C, D and E where the earlier released fragment A represents the shortest fragments and the later released fragment E represents the longest DNA fragments.

### F. Analysis of Released DNA Fragments.

Released DNA fragments can be directly used for analysis or additionally amplified by PCR using universal primers complementary to the adaptor sequences.

For sequencing applications, it is important that minimal number of errors is introduced during polymerase-mediated size-fractionation. Polymerases such as Taq DNA polymerase and Bst DNA polymerase are known to have low-fidelity of the DNA synthesis. To overcome potential problems, DNA strand created by nick-translation or strand-displacement synthesis can be destroyed when size fractionation is completed and only native strand used in the down-stream applications. To achieve this goal, dU base should be incorporated during DNA replacement synthesis, such that the synthesized DNA strand could be efficiently degraded by UDG enzyme and heating at 95° C., while original template DNA strand would survive such treatment. The template DNA strand of the size-fractionated polymer can be directly used for sequence analysis or after amplification by PCR using universal primers A an B and a high fidelity DNA polymerase such as Phusion DNA polymerase.

### IV. FRACTIONATION OF POLYNUCLEOTIDES BY SEQUENCE

Sequence-specific fractionation of polynucleotides is useful, for example, for DNA re-sequencing projects using NGS (e.g., for discovery of disease-associated genetic defects) and nucleic acid-based diagnostics.

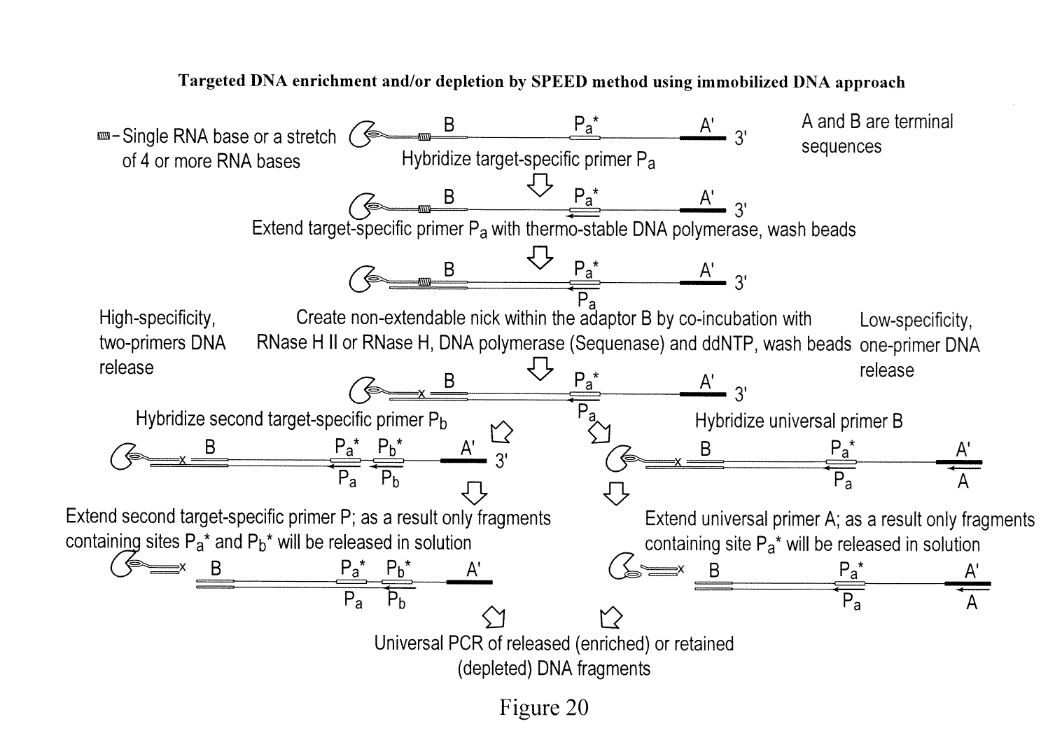

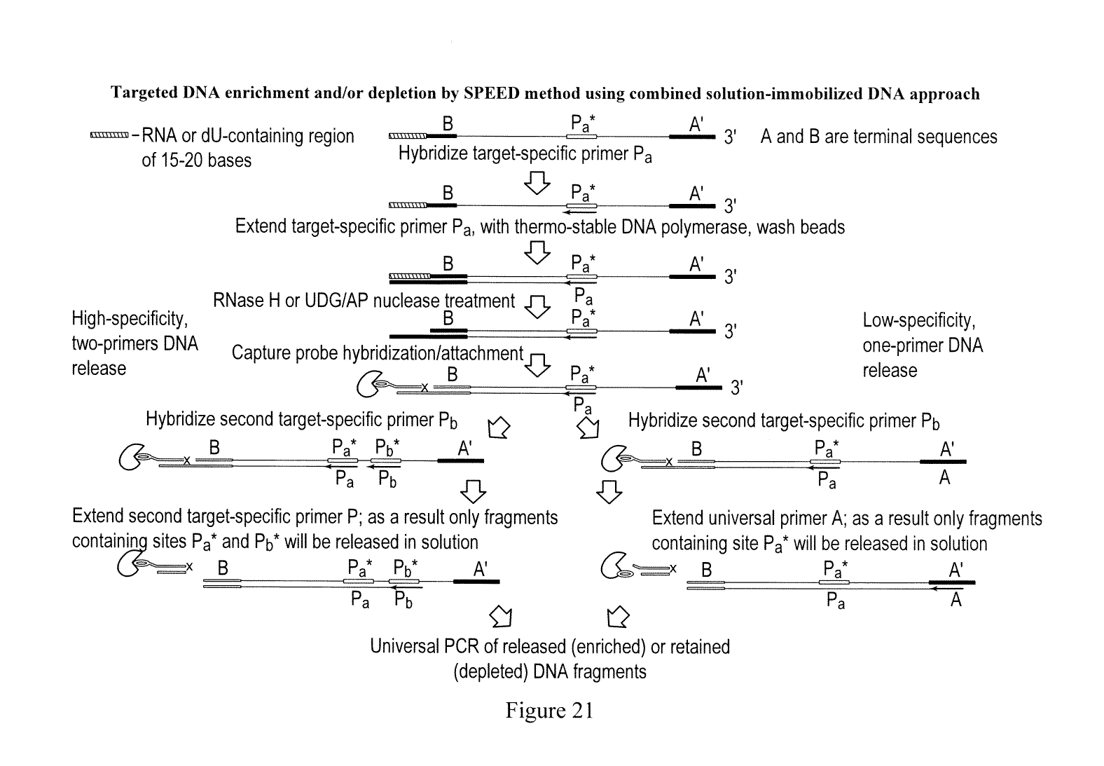

In various embodiments, the invention provides materials and methods for “DNA targeted enrichment/depletion by solid phase entrapment and enzymatic detachment” or DNA TED-SPEED″ or “multiplexed targeted enrichment/depletion by SPEED method” or “TED-PLEX-SPEED.” As illustrated in FIGS. 20 and 21 the following major steps generally occur in such methods although, as described herein, these steps (e.g., immobilization) need not necessarily occur in this order:

(1) Immobilization of single-stranded DNA fragments by their 5′-end;

(2) Hybridization and extension of a first SPEED (target-specific) primer complementary to target DNA. This process will convert targeted single-stranded DNA molecules into partially double-stranded DNA molecules;

(3) Creation of a break (e.g., gap) within a double-stranded end (proximal to the solid support) of the targeted DNA fragments using double-strand-specific nucleases such as a nickase or an RNAase H;

(4) Termination of the created break (e.g., gap) with a dideoxynucleotide using DNA polymerase, followed by a wash (in one embodiment, steps 3 and 4 are executed in a single reaction, for example, by mixing RNase H, polymerase (or terminal transferase (TdT)) and appropriate dideoxytriphoshate); and

(5) Hybridization and extension of a second SPEED primer located up-stream of the first SPEED primer.

As will be appreciated by a person of skill in the art and as shown in FIG. 21 the immobilization step may follow the initial primer hybridization and extension step. This type of immobilization may arise, for example, via hybridization of a labeled probe or capture probe to the 5′ end of the 5′ terminal sequence following the creation of a gap or break.

The second SPEED primer can be a target-specific primer or a universal sequence primer. DNA polymerization initiated by second primer would release targeted DNA molecules into solution when synthesized DNA strand reaches the break (e.g., gap) in the immobilized DNA strand. Separation and amplification of released DNA molecules results in an “enriched” fraction, while separation and amplification of retained DNA molecules results in a “depleted fraction.” FIGS. 20 and 21 further describe TED-SPEED DNA sequence enrichment and depletion.

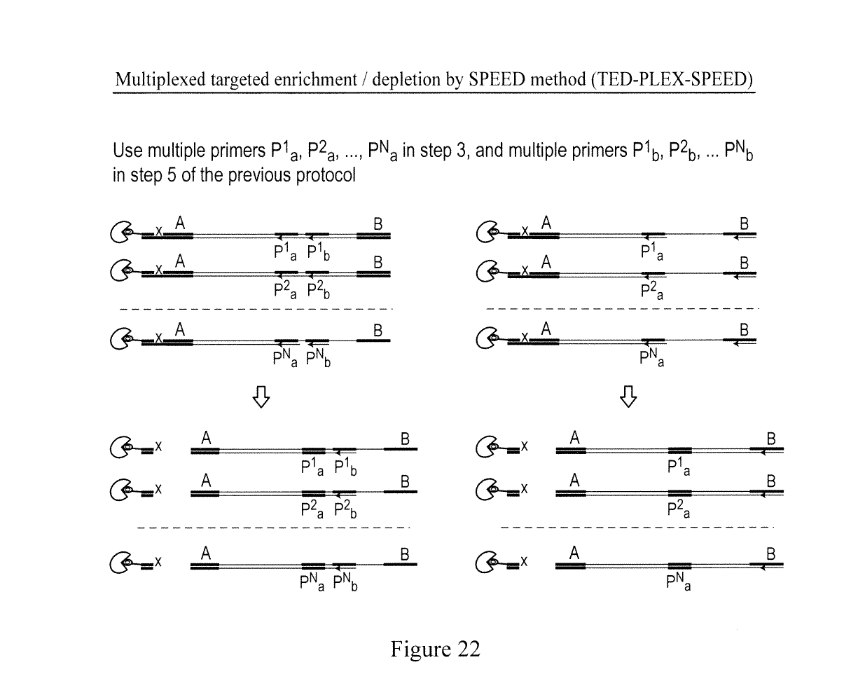

As will be readily appreciated by a person of skill in the art from FIGS. 20 and 21, hybridization and extension at stringent conditions (e.g., high temperature, low salt, etc.) of the first target-specific primer Pa facilitates creation of non-extendable nick or gap in the solid phase-bound adaptor B of only those DNA fragments that contain sequence Pa* complementary to primer sequence Pa. Hybridization and extension of the universal primer A would result in detachment of those immobilized DNA fragments that contain sequence Pa*. Detached fragments can be then amplified by PCR using universal primers A and B and, if so desired, analyzed further. It is very likely that the use of only one target-specific primer Pa would result in release and amplification of specific and non-specific DNA products. For this reason, use of second target-specific primer Pb for DNA detachment would result in substantial improvement of specificity of DNA isolation and amplification. It is expected that use of two target-specific primers Pa and Pb for DNA detachment would result in reduction or elimination of non-specific DNA products. In this case, PCR with two universal primers A and B would result in highly specific amplification of DNA fragments containing regions Pa and Pb (FIG. 22).

As will be readily appreciated by a person of skill in the art from FIGS. 20-22, hybridization and extension at stringent conditions (e.g., high temperature, low salt, etc.) of the first set of target-specific primers P1a, P2a, P3a, . . . , PNa facilitates creation of non-extendable nick or gap in the solid phase-bound adaptor B of only those DNA fragments that contain sequences P1a*, P2a*, P3a*, . . . , PNa* complementary to primer's sequences P1a, P2a, P3a, . . . , PNa. Hybridization and extension at stringent conditions of the second set of target-specific primers P1b, P2b, P3b, . . . , PNb results in detachment of multiple DNA fragments that contain corresponding sequences P1a* and P1b*, P2a* and P2b*, P3a* and P3b*, . . . , PNa* and PNb*. PCR with universal primers A and B would allow highly multiplexed and specific amplification of multiple DNA targets. Use of only one specific primer would result in substantially higher level of non-specifically released DNA fragments.

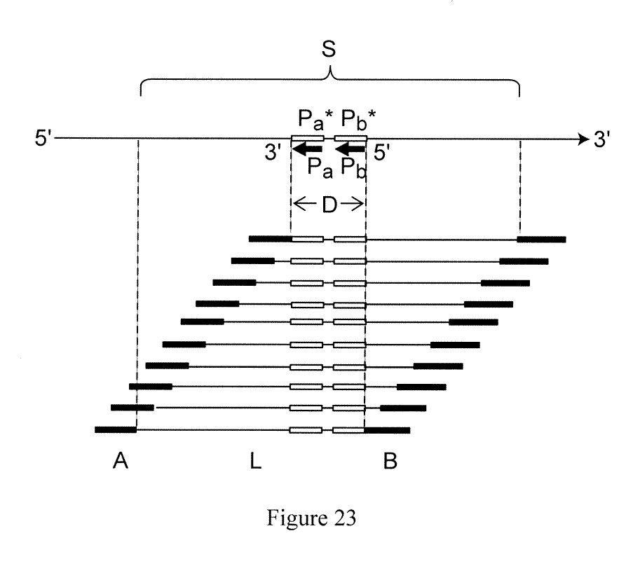

In still another embodiment, materials and methods are provided to enrich randomly fragmented DNA fragments. By way of example, FIG. 23 shows a genomic region “S” isolated by a two-primer capture-release method, where the size of region S is determined by formula: S=2L−D, where L is the library amplicon size, and D is the distance between the 3′ end of the primer Pa and the 5′ end of the primer Pb (for L=200 b and D=60 b, S=340 b).

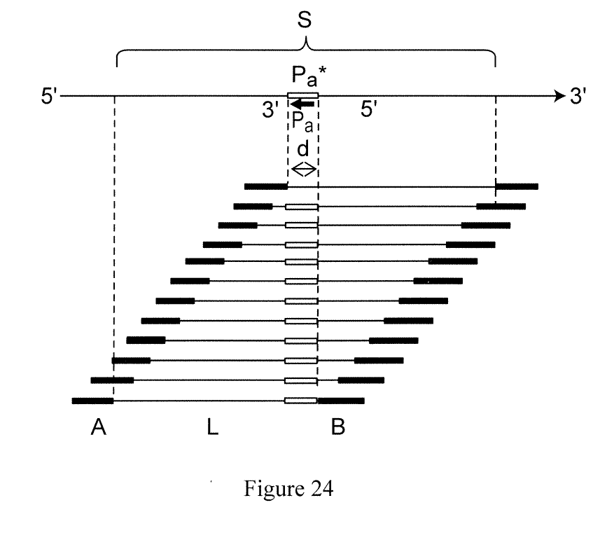

In a related embodiment, FIG. 24 shows a genomic region “S” isolated by a one-primer capture-release method. In this case, the size of region S is determined by formula: S=2L−d, where L is the library amplicon size, and d is the size of primer Pa (for L=200 b and D=20 b, S=380 b)

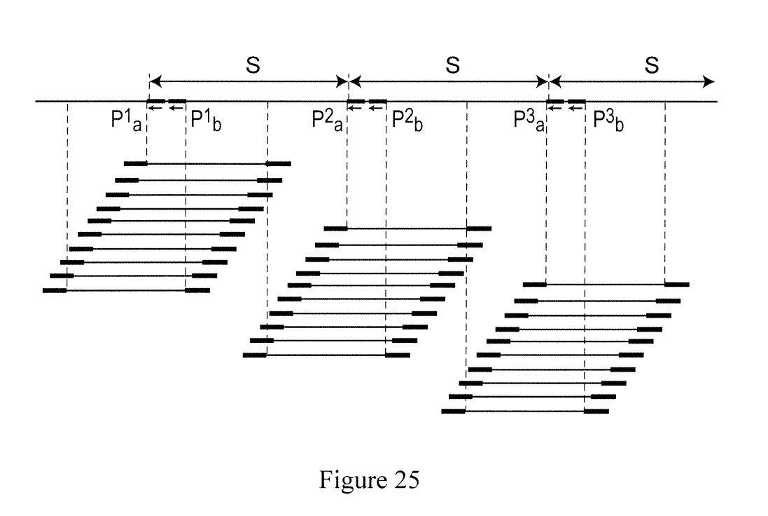

Primers may be designed for use in TED-PLEX-SPEED for creating a contig of library amplicons redundantly covering a large selected genomic region. Following FIGS. 23 and 24, primer pairs Pa and Pb for TED-PLEX-SPEED should be within a short distance D (˜20-60 b) to achieve maximal selected genomic region coverage (FIG. 25).

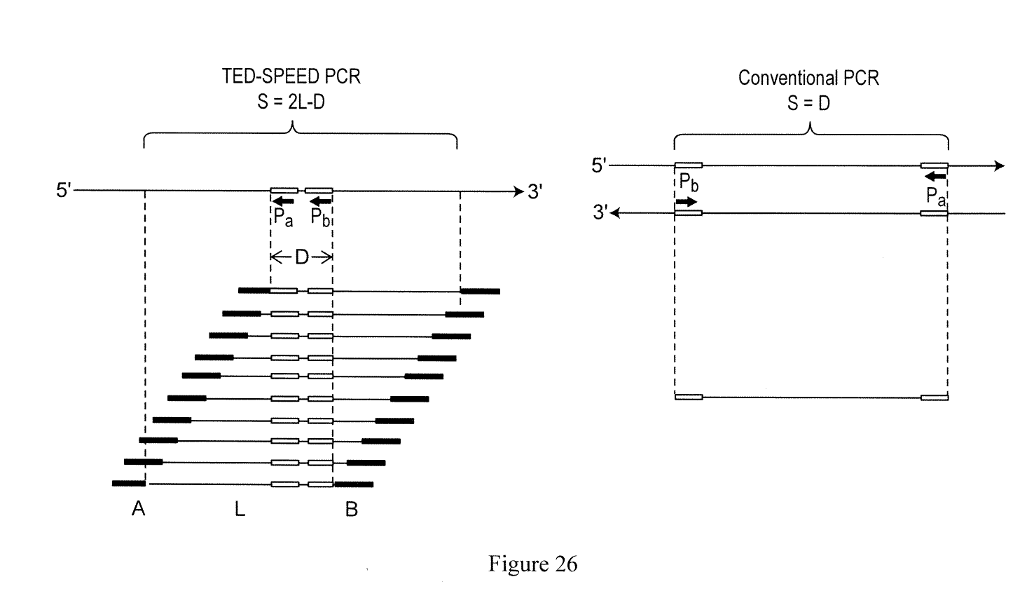

A comparison of TED-SPEED-mediated PCR and conventional PCR as tools for DNA generation for sequencing by NGS methods reveals the following characteristics. TED-SPEED-mediated PCR involves amplification by universal primers A and B; the amplified region is mostly determined by the size of the library L and minimally affected by the distance between specific detachment primers Pa and Pb; and there is no sequence bias for the ends of PCR amplicons. On the other hand, conventional PCR involves amplification by specific primers Pa and Pb; the amplified region is strictly determined by the distance between specific primers Pa and Pb; and the ends of PCR amplicons have the strongest sequence bias (FIG. 26).

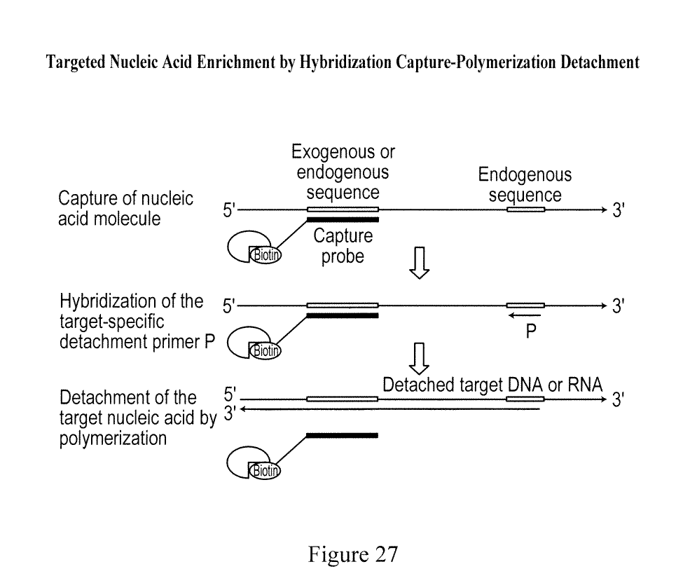

In still another embodiment, the invention provides materials and methods for targeted nucleic acid enrichment by hybridization capture-polymerization detachment, as illustrated In FIG. 27.

Of course, one of skill in the art would appreciate that the capture probe may be designed to hybridize to an adaptor sequence or to the target sequence.

### V. AMPLIFICATION AND MANIPULATION OF FRACTIONATED POLYNUCLEOTIDES

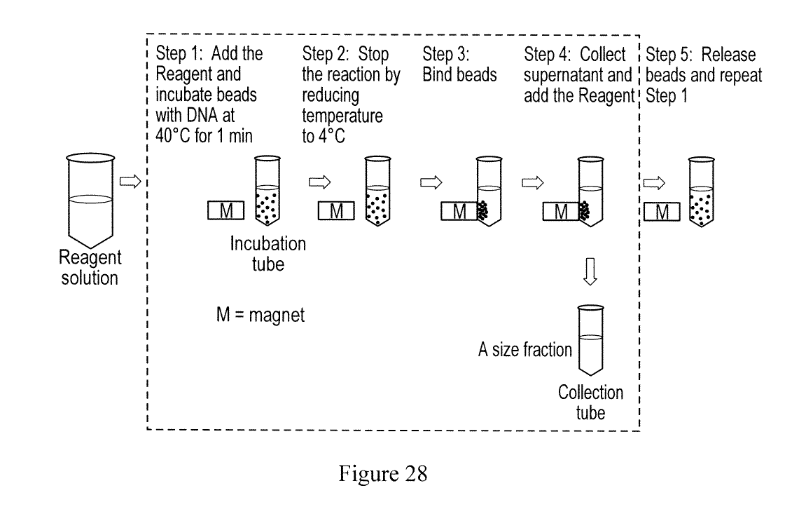

Various means of collecting fractionated polynucleotides are also contemplated by the present invention. By way of example, FIG. 28 illustrates the collection of a supernatant sample containing desired polynucleotides.

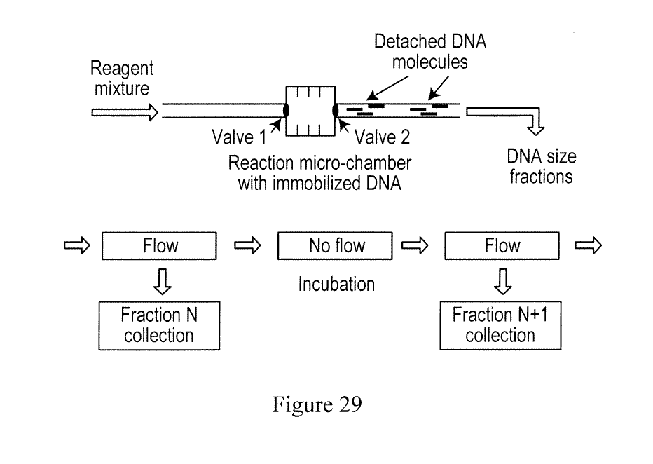

The worker of ordinary skill in the art will readily appreciate that any number of devices may be used in conjunction with the materials and methods provided herein. In one aspect, a DNA size sorter may also be used with the materials and methods provided herein (FIG. 29).

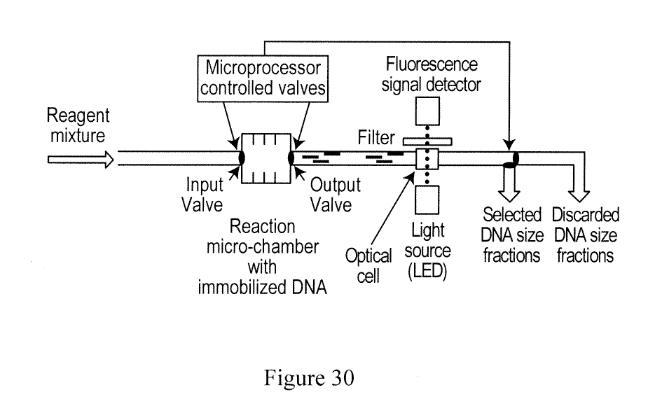

In another aspect, a DNA size analyzer and sorter may be used with the materials and methods provided herein (FIG. 30).

The worker of ordinary skill in the art will also readily appreciate that any number of molecular biological techniques and methods for the amplification of polynucleotides may be practiced in conjunction with the materials and methods provided herein. Such amplification methods include, but are not limited to, polymerase chain reaction (PCR), multiple-displacement amplification (MDA), rolling-circle amplification (RCA), Loop-Mediated Isothermal Amplification (LAMP), and the like.

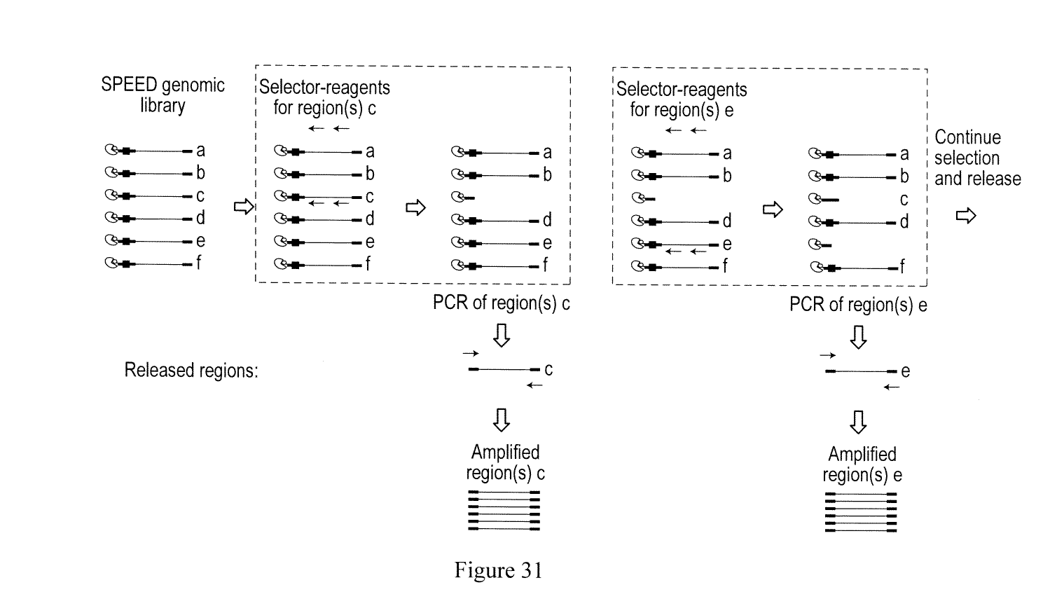

Indeed, one advantage of the present disclosure is the serial reusability of SPEED DNA libraries for targeted DNA isolation and amplification. For example, while conventional PCR allows a very limited number (e.g., one or two) of reactions per DNA template starting material, a SPEED DNA library allows an unlimited number of sequentially-released and amplified regions (e.g., target sequence). For example, in one aspect, the target sequence(s) (single or multiplexed) are detached and amplified individually, as shown in FIG. 31.

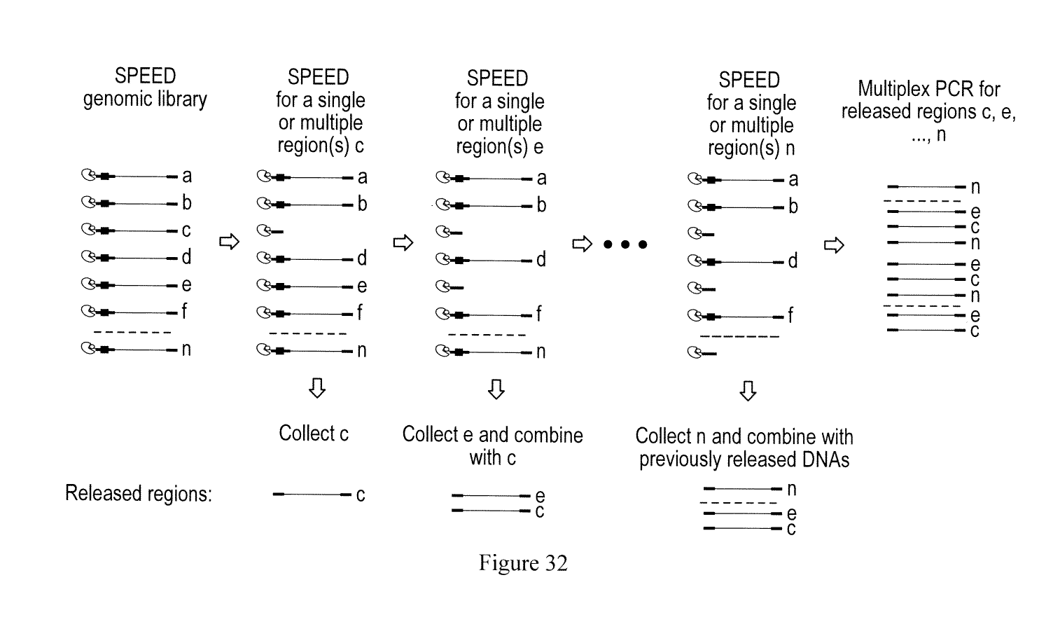

In another aspect, the target sequence(s) (single or multiplexed) are detached individually, but are pooled at the end and amplified in one multiplexed PCR reaction, as shown in FIG. 32.

### VI. COMPOSITIONS

Compositions are also contemplated by the invention. In various aspects compositions are provided comprising polynucleotides wherein the polynucleotide comprises a label, a single-stranded region, a double-stranded region, single-stranded or double-stranded 5′ and 3′ terminal sequences (e.g., adaptor sequences), modified bases, nuclease-resistant bases, and/or dideoxynucleotides as described herein. By way of example, a polynucleotide with at least one double-stranded region is provided, wherein the polynucleotide comprises a double-stranded 5′ terminal sequence comprising one or more nuclease resistant bases or one or more RNA residues, or a nick or gap. In another aspect, a polynucleotide with at least one double-stranded region is provided, wherein the polynucleotide comprises 5′ terminal sequence comprising one or more nuclease resistant bases or one or more RNA residues, or a nick or gap and a double-stranded 3′ terminal sequence with or without an extendable nick or gap. In still another aspect, a polynucleotide with at least one double-stranded region is provided, wherein the polynucleotide comprises 5′ terminal sequence comprising one or more nuclease resistant bases or one or more RNA residues, or a nick or gap, and a single-stranded 3′ terminal sequence.

### VII. KITS

The invention further provides kits comprising compositions described herein, as well as enzymes and reagents for carrying out the methods described herein. In one aspect, the kit comprises a composition of the invention packaged in a manner which facilitates its use in the methods of the invention. In one aspect, such a kit includes a composition described herein, packaged in a container such as a sealed bottle or vessel, with a label affixed to the container or included in the package that describes use of the composition in practicing the method. The kit may further include a device suitable for analyzing and/or collecting the composition.

### VIII. EXAMPLES

The Examples provided below illustrate the use of the materials and methods described herein.

### Example 1

### Targeted Enrichment and Amplification of Micro-Satellite (AC/TG)n—Repeat Containing DNA Fragments by One-Primer Capture-Release Method

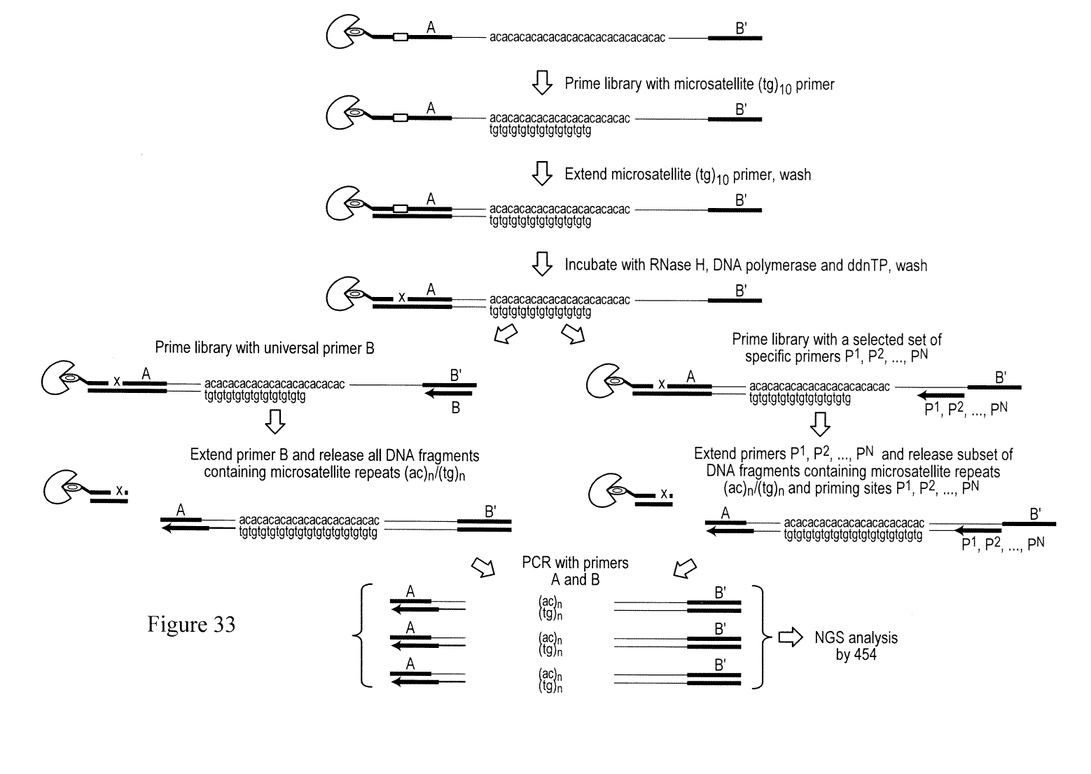

The following example provides a protocol for the targeted enrichment of micro-satellite (AC/TG)n—repeat containing DNA fragments by one-primer capture-release method. Fragments released by this protocol will contain repeats of different size and adjacent unique sequences. Using the NGS platform with long reads (e.g., Roche/454 GS FLX system) it is possible to determine unique sequences and the size of repeats and, as a result, perform genome-wide genotyping using repeats (˜2-3,000) as markers for gene-association studies (FIG. 33).

### Example 2

### Targeted Enrichment of Mutated K-Ras Codon

The following example provides a protocol for the targeted enrichment of a mutated K-ras codon. NGS represents a very promising approach for targeted re-sequencing and analysis of cancer-related genes such as K-ras.

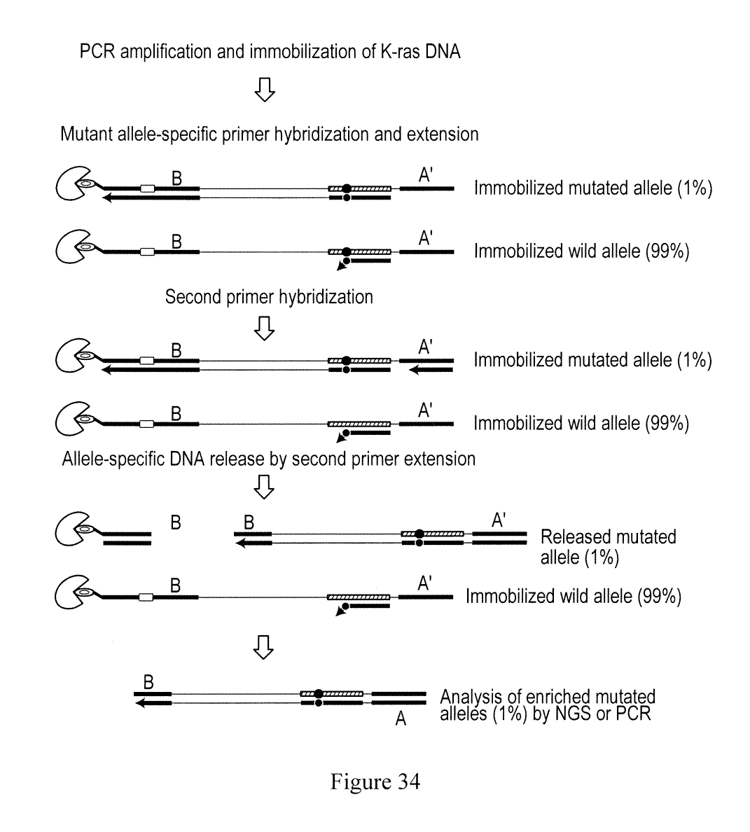

Mutations in K-ras codons 12 and 13 are important biomarkers for detection of colorectal, lung and many other cancers. They are also very important markers for predicting efficiency of EGFR-targeted antibody treatment in metastatic colorectal cancer. Frequently, surgical or biopsy samples contain only limited number of cells with K-ras mutations, so an enrichment of mutant alleles or a depletion of wild alleles can substantially improve detection of mutant alleles and allow multiplex analyses of larger numbers of samples, thereby dramatically reducing the cost of such analysis (FIG. 34).

### Example 3

### Targeted Depletion of Wild K-Ras Codon

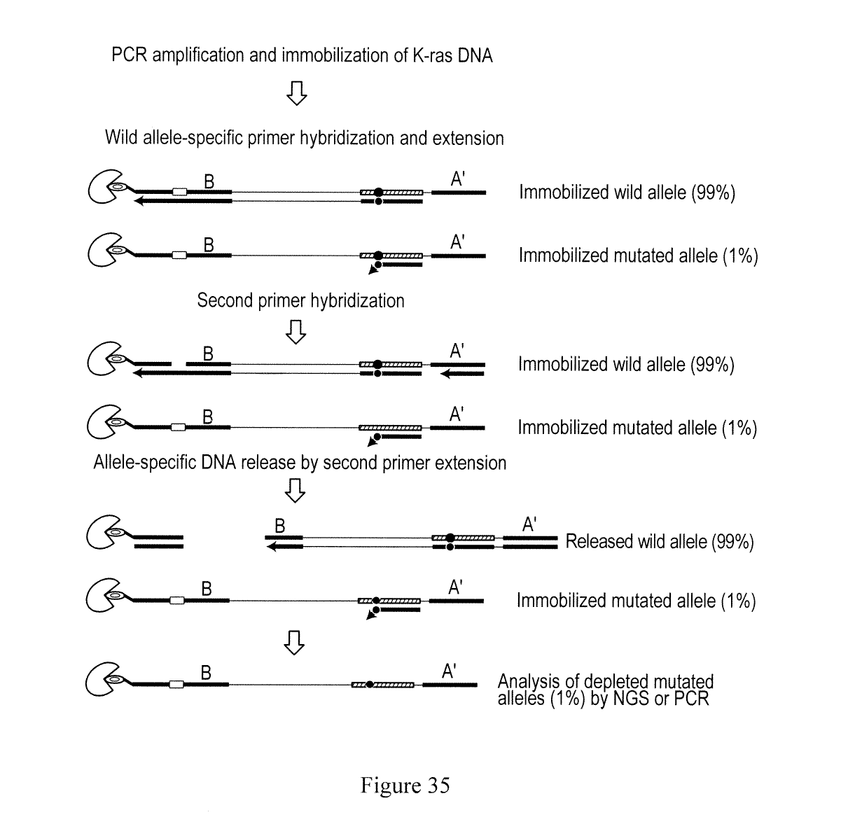

FIG. 35 provides a protocol for the targeted depletion of the wild-type K-ras codon.

### Example 4

### SPEED Size Separation of Lambda DNA PCR Fragments

This example demonstrates that SPEED Size Separation can be used to release and isolate immobilized DNA fragments of a specific size by incubating solid phase with SPEED reaction mix.

TABLE 1
List of Oligonucleotide Sequences

| | | | SEQ |
|---|---|---|---|
| Oligo ID | Description | Sequence (5′-3′) | ID NO |
| 10-0117 | Forward 100 bp | ACTCCTCAACGCACGAAACACGACCGAATATCCTGCATTCCCGAACC | 1 |
| 10-0118 | Forward 200 bp | ACTCCTCAACGCACGAAACACGACCCTTCAAAAGGCCACCTGTTACTG | 2 |
| 10-0119 | Forward 300 bp | ACTCCTCAACGCACGAAACACGACCTCCTTGGGTCCCTGTAGCAG | 3 |
| 10-0120 | Forward 500 bp | ACTCCTCAACGCACGAAACACGACCGTGTCTTCTGCTTGATTCCTCTG | 4 |
| 10-0121 | Forward 700 bp | ACTCCTCAACGCACGAAACACGACCTGGCAGCGACATGGTTTGTTG | 5 |
| 10-0122 | Forward 1000 bp | ACTCCTCAACGCACGAAACACGACCGCAATTATGGTTTCTCCGCCAAG | 6 |
| 10-0123 | Lambda Reverse | CAGCACAGGAACCAACCACAGCAATAAGGGAGACTTTGCGATGTACTTGAC | 7 |
| 10-0124 | SPEED SIZE | ACTCCTCAACGCACGAAACArCrGrArCC | 8 |
| | Forward | | |
| 10-0442 | SPEED SIZE | Dual biotin-AAAACAGCACAGGAACCAACCACAGCAAUAAG | 9 |
| | Reverse | | |
| 10-0370 | Forward 50 bp | ACTCCTCAACGCACGAAACACGACCCGTCGCTGGCGTGCGTTCC | 10 |
| 10-0358 | Forward 150 bp | ACTCCTCAACGCACGAAACACGACCCCTTTACCGCTGATTCGTGGAAC | 11 |
| 10-0359 | Forward 250 bp | ACTCCTCAACGCACGAAACACGACCGGTTAGGGGGTAAATCCCGGC | 12 |
| 10-0360 | Forward 350 bp | ACTCCTCAACGCACGAAACACGACCGGCCTGTATAGCTTCAGTGATTGCG | 13 |
| 10-0361 | Forward 400 bp | ACTCCTCAACGCACGAAACACGACCGCTGTCAGAGGCTTGTGTTTGTG | 14 |
| 10-0362 | Forward 450 bp | ACTCCTCAACGCACGAAACACGACCCGTAGCGATCAAGCCATGAATG | 15 |
| 10-0363 | Forward 550 bp | ACTCCTCAACGCACGAAACACGACCCTTCAAGTGGAGCATCAGGCAG | 16 |
| 10-0364 | Forward 600 bp | ACTCCTCAACGCACGAAACACGACCGTATCCATTGAGCATTGCCGC | 17 |
| 10-0365 | Forward 650 bp | ACTCCTCAACGCACGAAACACGACCGGAATGCATCGCTCAGTGTTGATC | 18 |
| 10-0366 | Forward 750 bp | ACTCCTCAACGCACGAAACACGACCCGTCAGCCGTAAGTCTTGATC | 19 |
| 10-0367 | Forward 800 bp | ACTCCTCAACGCACGAAACACGACCGCCAACATGGTGATGATTCTGC | 20 |
| 10-0371 | Forward 850 bp | ACTCCTCAACGCACGAAACACGACCCTCGTTGCCCGGTAACAACAGCC | 21 |
| 10-0372 | Forward 900 bp | ACTCCTCAACGCACGAAACACGACCCGACATAAAGATATCCATCTACG | 22 |
| 10-0373 | Forward 950 bp | ACTCCTCAACGCACGAAACACGACCCAATATGCAATGCTGTTGGG | 23 |
| 11-0057 | SPEED | Dual biotin-AAAACAGCACAGGAACCAACCArCrArGrCAATAAG | 24 |
| | Sequence | | |
| | Reverse | | |

### Method:

DNA fragments of sizes 50 to 1000 bp (in 50 bp increments) were PCR amplified from 10 ng of Lambda DNA (NEB N3011S) using Bio-Rad iQ SYBR-Green Supermix (170-8882) with the universal reverse primer (oligo 10-0123) and the appropriate forward primer (see table 1) for the desired size fragment. Amplification was performed in a Bio-Rad CFX96 thermocycler using a 3-step amplification protocol (see table 2). Following amplification the reactions were diluted 1:400 and then used as templates in a second round of PCR using iQ SYBR Green Super mix and universal primers to add the forward and reverse adaptors for SPEED Size Separation (Primers 10-0124 and 10-0442).

TABLE 2
Thermocycling Protocol for all rounds of PCR

| Step | Temp (deg C.) | Time | | |
|---|---|---|---|---|
| Initial Denaturation | 95 | 3 m | | |
| Denaturation | 95 | 15 | | |
| Annealing | 60 | 20 | {close oversize brace} | 25-30 cycles |
| Extension | 70 | 60 | | |

After the second round of amplification, the resulting products were purified using the Qiagen PCR DNA Clean-Up Kit (Cat #28106) and quantified by Nanodrop spectrophotometric analysis. A solution with equil mass of all fragments for SPEED Size Separation was then prepared to final concentrations of 30 ng/uL in nuclease free water.

The solution of fragments was immobilized on JSR Micro MS300 streptavidin coated magnetic beads at a ratio of 250 ug beads to 100 ng of DNA by incubating together in 1× bead wash buffer (10 mM Tris-HCL (pH7.4) with 0.5 mM EDTA, 1M NaCl, 0.05% Tween20) for 20 minutes at room temperature while on a tube rotator. Following immobilization, the bead solution was magnetized to pellet the beads and the supernatant was removed. The beads were then washed twice with 100 ul of 1× bead wash buffer and twice with 100 ul of 1× bead storage buffer (10 mM Tris-HCl (pH 8.0) with 0.1 mM EDTA, 10 mM NaCl) beads could then be stored at 4 degrees Celsius (10 mg/mL) until needed.

SPEED Size Separation was performed using the prepared beads as follows. Nicks (one nick-polymerase non-extendable, and one-polymerase extendable) were introduced in adaptors A and B (created by primers 10-0442 and 10-0124 in a second round of PCR) by incubation with 0.5-1.0 units of USER (NEB M5505L) and 2.5-5 units of RNase H(NEB M0297S) at 37 degrees C. for half an hour. Incubation with USER enzyme mix created a non-extendable single-base gap at the position of dU base within immobilized adaptor A (sequence 10-0442), while incubation with RNase H created an extendable nick in the middle of the RNA stretch rCrGrArC of sequence 10-0124. During this time, a master mix of SPEED Size reagents was prepared containing 0.5 units/uL of Taq polymerase (NEB M0320S), 1 mM dNTP mix (Invitrogen 18427-013), and 5 mM MgCl2 in 1×NEB Taq reaction buffer. Following incubation with RNase H and USER the beads were pelleted by magnetic-stand and the supernatant was removed. The beads were gently washed twice with 50 uL of 1× Taq reaction buffer and then resuspended in 20 uL 1× Taq reaction buffer containing Taq DNA polymerase and all master mix reagents except dNTPs. The bead suspension and master mix were placed into a block heated to 50 C and allowed to reach thermal equilibrium. The beads were then pelleted by sliding a magnet into place behind the heated block and the supernatant was removed and replaced with 20 uL of master mix and the beads were resuspended by pipetting up and down after removal of the magnet. This step can be repeated as many times as needed for the number of time points desired. Time points were taken in 1 minute intervals but can be adjusted as necessary to obtain a desired size fraction.

After obtaining size fractions they were analyzed using the Agilent 2100 Bioanalyzer and a DNA 1000 Kit (Agilent 5065-4449) and by separation on a 4-12% TBE-Acrylamide gel (Invitrogen EC62352BOX) which was stained with SYBRGold (Invitrogen S11494) and visualized on a transilluminator.

### Results:

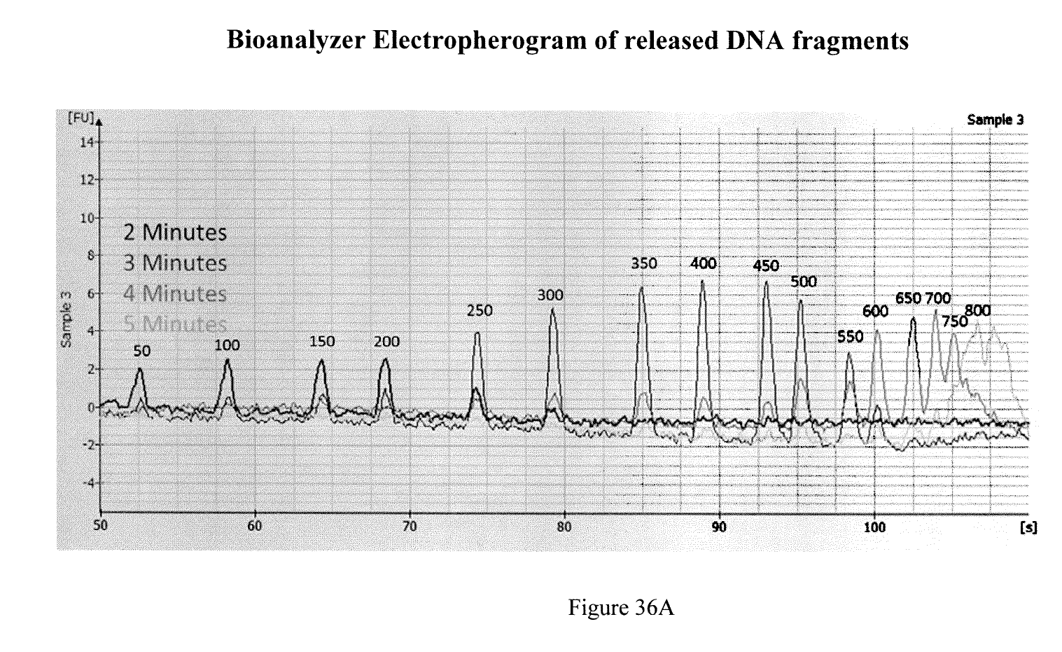

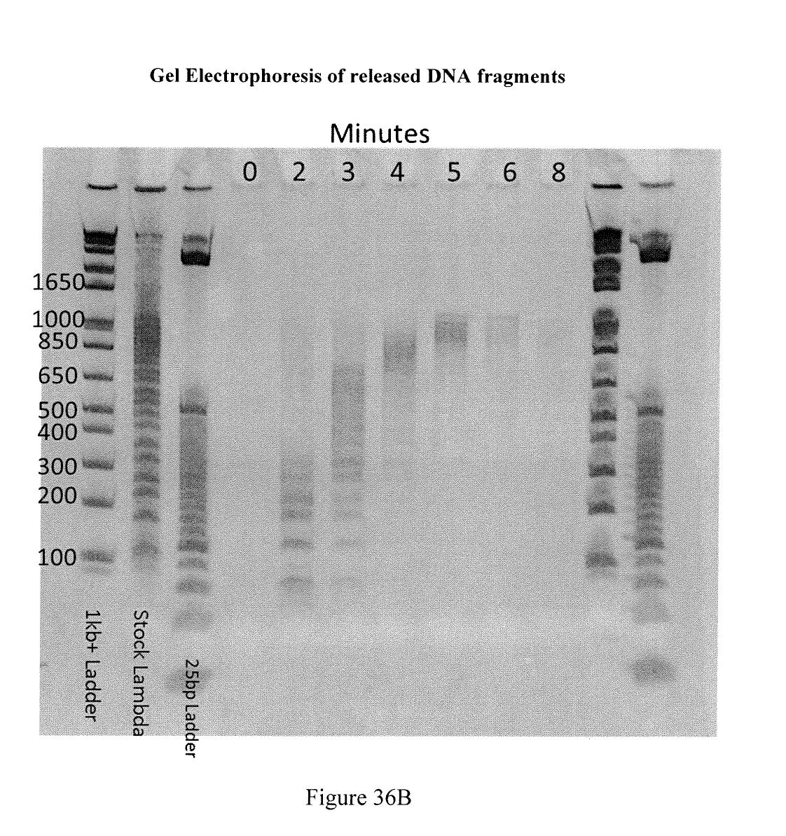

Bioanalyzer 2100 Electrophoregram and gel electrophoresis of released DNA fragments are shown on FIG. 36A (time points 2, 3, 4 and 5 min) and FIG. 36B (time points 0, 2, 3, 4, 5, 6 and 8 min), respectively. Gel electrophoretic and Bioanalyzer 2100 analysis of products of SPEED Size selection indicate that fragments of increasing size are released from the solid phase through successive incubations with reaction mix thus providing a proof of concept of DNA size selection by solid phase immobilization-enzymatic release approach.

### Example 5

### SPEED Sequence Selection of Lambda DNA PCR Fragments

This Examples demonstrates that SPEED Sequence Selection Method can be used to release and isolate DNA fragments with a desired sequence composition.

TABLE 1
List of Oligonucleotide Sequences

| | | | SEQ |
|---|---|---|---|
| Oligo ID | Description | Sequence (5′-3′) | ID NO |
| 10-0117 | Forward 100 bp | ACTCCTCAACGCACGAAACACGACCGAATATCCTGCATTCCCGAACC | 1 |
| 10-0118 | Forward 200 bp | ACTCCTCAACGCACGAAACACGACCCTTCAAAAGGCCACCTGTTACTG | 2 |
| 10-0119 | Forward 300 bp | ACTCCTCAACGCACGAAACACGACCTCCTTGGGTCCCTGTAGCAG | 3 |
| 10-0120 | Forward 500 bp | ACTCCTCAACGCACGAAACACGACCGTGTCTTCTGCTTGATTCCTCTG | 4 |
| 10-0121 | Forward 700 bp | ACTCCTCAACGCACGAAACACGACCTGGCAGCGACATGGTTTGTTG | 5 |
| 10-0122 | Forward 1000 bp | ACTCCTCAACGCACGAAACACGACCGCAATTATGGTTTCTCCGCCAAG | 6 |
| 10-0123 | Lambda Reverse | CAGCACAGGAACCAACCACAGCAATAAGGGAGACTTTGCGATGTACTTGAC | 7 |
| 10-0124 | SPEED SIZE | ACTCCTCAACGCACGAAACArCrGrArCC | 8 |
| | Forward | | |
| 10-0442 | SPEED SIZE | Dual biotin-AAAACAGCACAGGAACCAACCACAGCAAUAAG | 9 |
| | Reverse | | |
| 10-0370 | Forward 50 bp | ACTCCTCAACGCACGAAACACGACCCGTCGCTGGCGTGCGTTCC | 10 |
| 10-0358 | Forward 150 bp | ACTCCTCAACGCACGAAACACGACCCCTTTACCGCTGATTCGTGGAAC | 11 |
| 10-0359 | Forward 250 bp | ACTCCTCAACGCACGAAACACGACCGGTTAGGGGGTAAATCCCGGC | 12 |
| 10-0360 | Forward 350 bp | ACTCCTCAACGCACGAAACACGACCGGCCTGTATAGCTTCAGTGATTGCG | 13 |
| 10-0361 | Forward 400 bp | ACTCCTCAACGCACGAAACACGACCGCTGTCAGAGGCTTGTGTTTGTG | 14 |
| 10-0362 | Forward 450 bp | ACTCCTCAACGCACGAAACACGACCCGTAGCGATCAAGCCATGAATG | 15 |
| 10-0363 | Forward 550 bp | ACTCCTCAACGCACGAAACACGACCCTTCAAGTGGAGCATCAGGCAG | 16 |
| 10-0364 | Forward 600 bp | ACTCCTCAACGCACGAAACACGACCGTATCCATTGAGCATTGCCGC | 17 |
| 10-0365 | Forward 650 bp | ACTCCTCAACGCACGAAACACGACCGGAATGCATCGCTCAGTGTTGATC | 18 |
| 10-0366 | Forward 750 bp | ACTCCTCAACGCACGAAACACGACCCGTCAGCCGTAAGTCTTGATC | 19 |
| 10-0367 | Forward 800 bp | ACTCCTCAACGCACGAAACACGACCGCCAACATGGTGATGATTCTGC | 20 |
| 10-0371 | Forward 850 bp | ACTCCTCAACGCACGAAACACGACCCTCGTTGCCCGGTAACAACAGCC | 21 |
| 10-0372 | Forward 900 bp | ACTCCTCAACGCACGAAACACGACCCGACATAAAGATATCCATCTACG | 22 |
| 10-0373 | Forward 950 bp | ACTCCTCAACGCACGAAACACGACCCAATATGCAATGCTGTTGGG | 23 |
| 11-0057 | SPEED | Dual biotin-AAAACAGCACAGGAACCAACCArCrArGrCAATAAG | 24 |
| | Sequence | | |
| | Reverse | | |

### Method:

DNA fragments of sizes 50 to 1000 bp (in 50 bp increments) were PCR amplified from 10 ng of Lambda DNA (NEB N3011S) using Bio-Rad iQ SYBR-Green Supermix (170-8882) with the universal reverse primer (oligo 10-0123) and the appropriate forward primer (see table 1) for the desired size fragment. Amplification was performed in a Bio-Rad CFX96 thermocycler using a 3-step amplification protocol (see table 2). Following amplification the reactions were diluted 1:400 and then used as templates in a second round of PCR using iQ SYBR Green Super mix and universal primers to add the forward and reverse adaptors for SPEED Sequence (Primers 10-0124 and 11-0057).

TABLE 2
Thermocycling Protocol for all rounds of PCR

| Step | Temp (deg C.) | Time | | |
|---|---|---|---|---|
| Initial Denaturation | 95 | 3 m | | |
| Denaturation | 95 | 15 | | |
| Annealing | 60 | 20 | {close oversize brace} | 25-30 cycles |
| Extension | 70 | 60 | | |

After the second round of amplification, the resulting products were purified using the Qiagen PCR DNA Clean-Up Kit (Cat #28106) and quantified by Nanodrop spectrophotometric analysis. An equimass solution of all fragments for SPEED Sequence Selection was then prepared to final concentrations of 30 ng/uL in nuclease free water.

The solution of fragments was immobilized on JSR Micro MS300 streptavidin coated magnetic beads at a ratio of 250 ug beads to 100 ng of DNA by incubating together in 1× bead wash buffer (10 mM Tris-HCL (pH7.4) with 0.5 mM EDTA, 1M NaCl, 0.05% Tween20) for 20 minutes at room temperature while on a tube rotator. Following immobilization, the bead solution was magnetized to pellet the beads and the supernatant was removed. The beads were then washed twice with 100 ul of 1× bead wash buffer and twice with 100 ul of 1× bead storage buffer (10 mM Tris-HCl (pH 8.0) with 0.1 mM EDTA, 10 mM NaCl) beads could then be stored at 4 degrees Celsius (10 mg/mL) until needed.

SPEED Sequence selection was performed using the prepared beads as follows. Immobilized DNA was heat denatured to remove the complimentary, non-biotinylated strand from the beads. This was done by heating the beads for 1 minute in 1× bead storage buffer and then quickly pelleting the beads and removing the complementary DNA solution before reannealing could occur. The first sequence specific oligo-primer 10-0120 and Taq DNA polymerase were then used to create a double-stranded region in 11 selected DNA fragments (1× Taq reaction buffer containing 5 mM MgC12 1 mM dNTPs 20 pmols oligo 10-120, 10 U Taq). This reaction was performed by incubating at 95 C for 15 s followed by 60 C for 20 s and 70 C for 5 minutes. The beads were then gently washed with 1× bead wash buffer and 1× Taq buffer and resuspended in 1×NEB RNase H buffer. 5 units of RNase H were added to the bead suspension and incubated at 37 C for 30 minutes. The beads were pelleted, and washed twice with 1× bead storage buffer. This treatment created a nick within RNA/DNA duplex region of the immobilized adaptor A sequence (11-0057). In order to ensure that the nick created by RNase H would not be extendable by a DNA polymerase, a nucleotide blocking reaction was performed. For this purpose the pelleted beads were resuspended in 20 uL of 1× Sequenase reaction buffer containing 1 mM each of dideoxy-ATP, CTP, and GTP (USB 77126), and 3.25 units of Sequenase 2.0 enzyme (USB 70775Y) and incubated at 37 C for 10 minutes.

Release of the desired sequences was performed by a second polymerase reaction as follows. The beads were pelleted and washed gently twice with 1× Taq reaction buffer. They were then resuspended in 20 uL of 1× Taq reaction buffer containing 5 mM MgCl2, 1 mM dNTPs, 20 pmols of either primer 10-367 (800 bp sequence) or 10-0364 (600 bp sequence), and 10 units of Taq polymerase. The beads were then incubated for 10 minutes at 50 C, pelleted and the solution which contained the released DNA fragments was transferred to a microfuge tube. The collected solutions were diluted 1:1 with formamide buffer and heated to 95 C prior to separation on a 6% TBU-Acrylamide gel (Invitrogen EC68652BOX) which was stained with SYBRGold (Invitrogen S11494) and visualized on a transilluminator.

### Results:

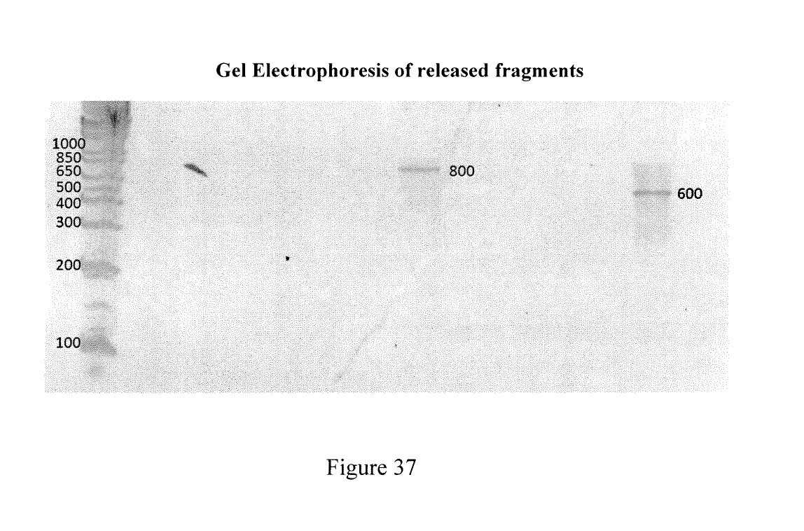

Gel electrophoresis of DNA fragments released by SPEED Sequence Selection protocol is shown on FIG. 37. Electrophoretic analysis indicate that only 800 bp and 600 bp fragments containing the desired sequences as defined by the two extension primers 10-367 and 10-0364 were released from the beads.

1. A method of preparing a desired polynucleotide comprising contacting a labeled polynucleotide having a double-stranded region comprising a first strand and a second strand wherein either the first strand or second strand is labeled and immobilized with an enzyme or enzyme mixture under conditions wherein the interaction of the first strand with the second strand is reduced, thereby resulting in dissociation of the second strand from the first strand, wherein either an unlabeled first strand or unlabeled second strand is the desired polynucleotide.

2-92. (canceled)

93. A composition comprising a labeled polynucleotide having a double-stranded region, the labeled polynucleotide comprising a first strand having a 5′ terminus and a 3′ terminus and a second strand having a 5′ terminus and a 3′ terminus, the labeled polynucleotide further comprising a double-stranded target sequence between a double-stranded 5′ terminal sequence and a double-stranded 3′ terminal sequence, the 5′ terminal sequence including the 5′ terminus of the first strand and a sequence adjacent and the 3′ terminus of the second strand and a sequence adjacent, the 3′ terminal sequence including the 3′ terminus of the first strand and a sequence adjacent and the 5′ terminus of the second strand and a sequence adjacent, the 5′ terminal sequence comprising one or more nuclease-resistant bases or one or more RNA residues.

94. A composition comprising a labeled polynucleotide having a double-stranded region, the labeled polynucleotide comprising a first strand having a 5′ terminus and a 3′ terminus and a second strand having a 5′ terminus and a 3′ terminus, the labeled polynucleotide further comprising a double-stranded target sequence between a double-stranded 5′ terminal sequence and a single stranded 3′ terminal sequence, the 5′ terminal sequence including the 5′ terminus of the first strand and a sequence adjacent and the 3′ terminus of the second strand and a sequence adjacent, the 3′ terminal sequence including the 3′ terminus of the first strand and a sequence adjacent and the 5′ terminus of the second strand and a sequence adjacent, the 5′ terminal sequence comprising one or more nuclease-resistant bases or one or more RNA residues.

95. A method of preparing a desired polynucleotide comprising the steps of:
a) preparing a labeled polynucleotide, wherein the labeled polynucleotide comprises a first strand having a 5′ terminus and a 3′ terminus and a second strand having a 5′ terminus and a 3′ terminus, and wherein the labeled polynucleotide comprises a double-stranded target sequence between a double-stranded 5′ terminal sequence located proximal to the label, and a double-stranded 3′ terminal sequence located distal to the label, the double-stranded 5′ terminal sequence including the 5′ terminus of the first strand and a sequence adjacent and the 3′ terminus of the second strand and a sequence adjacent, the double-stranded 3′ terminal sequence including the 3′ terminus of the first strand and a sequence adjacent and the 5′ terminus of the second strand and a sequence adjacent;
b) immobilizing the labeled polynucleotide, said immobilizing comprising attaching the 5′ end of the double-stranded 5′ terminal sequence on the first strand to a solid phase binding partner or, in the alternative, attaching the 3′ end of the double-stranded 5′ terminal sequence on the second strand to a solid phase binding partner;
c) optionally introducing a nick or gap at the 5′ end of the double-stranded 5′ terminal sequence on the first strand proximal to the solid phase binding partner in the case of 5′ end attachment in step b);
d) introducing a nick or gap at the 5′ end of the double-stranded 3′ terminal sequence on the second strand distal to the solid phase binding partner; thereby creating an extendable break in the 5′ end of the double-stranded 3′ terminal sequence on the second strand distal to the solid phase binding partner; and
f) contacting the labeled polynucleotide with an enzyme with 5′-3′ exonuclease and polymerase activity under conditions wherein the second strand of the labeled polynucleotide is degraded up to a non-degradable location and a new second strand is synthesized up to a non-extendable location, at which point the desired polynucleotide is separated from the labeled polynucleotide.

96. A method of preparing a desired polynucleotide comprising the steps of:
a) preparing a labeled polynucleotide, wherein the labeled polynucleotide comprises a first strand having a 5′ terminus and a 3′ terminus and a second strand having a 5′ terminus and a 3′ terminus, and wherein the labeled polynucleotide comprises a double-stranded target sequence between a double-stranded 5′ terminal sequence located proximal to the label, and a double-stranded 3′ terminal sequence located distal to the label, the double-stranded 5′ terminal sequence including the 5′ terminus of the first strand and a sequence adjacent and the 3′ terminus of the second strand and a sequence adjacent, the double-stranded 3′ terminal sequence including the 3′ terminus of the first strand and a sequence adjacent and the 5′ terminus of the second strand and a sequence adjacent;
b) immobilizing the labeled polynucleotide, said immobilizing comprising attaching the 5′ end of the double-stranded 5′ terminal sequence on the first strand to a solid phase binding partner or, in the alternative, attaching the 3′ end of the double-stranded 5′ terminal sequence on the second strand to a solid phase binding partner;
c) introducing a nick or gap at the 5′ end of the double-stranded 5′ terminal sequence on the first strand proximal to the solid phase binding partner;
d) introducing a nick or gap at the 5′ end of the double-stranded 3′ terminal sequence on the second strand distal to the solid phase binding partner; and
e) contacting the labeled polynucleotide with an enzyme with 5′-3′ exonuclease and polymerase activity under conditions wherein the first strand and the second strand of the labeled polynucleotide are degraded, and a new first strand and a new second strand are synthesized, up to an enzyme collision location, at which point the desired polynucleotide is separated from the labeled polynucleotide.

97. A method of preparing a desired polynucleotide comprising the steps of:
a) preparing a labeled polynucleotide, wherein the labeled polynucleotide comprises a first strand having a 5′ terminus and a 3′ terminus and a second strand having a 5′ terminus and a 3′ terminus, and wherein the labeled polynucleotide comprises a double-stranded target sequence between a double-stranded 5′ terminal sequence located proximal to the label, and a double-stranded 3′ terminal sequence located distal to the label, the double-stranded 5′ terminal sequence including the 5′ terminus of the first strand and a sequence adjacent and the 3′ terminus of the second strand and a sequence adjacent, the double-stranded 3′ terminal sequence including the 3′ terminus of the first strand and a sequence adjacent and the 5′ terminus of the second strand and a sequence adjacent;
b) immobilizing the labeled polynucleotide, said immobilizing comprising attaching the 5′ end of the double-stranded 5′ terminal sequence on the first strand to a solid phase binding partner or, in the alternative, attaching the 3′ end of the double-stranded 5′ terminal sequence on the second strand to a solid phase binding partner;
c) optionally introducing a nick or gap at the 5′ end of the double-stranded 5′ terminal sequence on the first′ strand proximal to the solid phase binding partner in the case of 5′ end attachment in step b);
d) introducing nick or gap at the 5′ end of the double-stranded 3′ terminal sequence on the second′ strand distal to the solid phase binding partner; and
e) contacting the labeled polynucleotide with an enzyme with polymerase activity under conditions wherein the second strand of the labeled polynucleotide is displaced, and a new second strand is synthesized up to a non-extendable location, at which point the desired polynucleotide is separated from the labeled polynucleotide.

98. A method of preparing a desired polynucleotide comprising the steps of:
a) preparing a labeled polynucleotide, wherein the labeled polynucleotide comprises a first strand having a 5′ terminus and a 3′ terminus and a second strand having a 5′ terminus and a 3′ terminus, and wherein the labeled polynucleotide comprises a double-stranded target sequence between a double-stranded 5′ terminal sequence located proximal to the label, and a double-stranded 3′ terminal sequence located distal to the label, the double-stranded 5′ terminal sequence including the 5′ terminus of the first strand and a sequence adjacent and the 3′ terminus of the second strand and a sequence adjacent, the double-stranded 3′ terminal sequence including the 3′ terminus of the first strand and a sequence adjacent and the 5′ terminus of the second strand and a sequence adjacent;
b) immobilizing the labeled polynucleotide, said immobilizing comprising attaching the 5′ end of the double-stranded 5′ terminal sequence on the first strand to a solid phase binding partner or attaching the 3′ end of the double-stranded 5′ terminal sequence on the second strand to a solid phase binding partner;
c) introducing nick or gap at the 5′ end of the double-stranded 5′ terminal sequence on the first strand proximal to the solid phase binding partner;
d) introducing nick or gap at the 5′ end of the double-stranded 3′ terminal sequence on the second strand distal to the solid phase binding partner; and
e) contacting the labeled polynucleotide with an enzyme with polymerase activity under conditions wherein the first strand and second strand of the labeled polynucleotide are displaced, and a new first strand and a new second strand are synthesized up to an enzyme collision location, at which point the desired polynucleotide is separated from the labeled polynucleotide.

99. A method of preparing a desired polynucleotide comprising the steps of:
a) preparing a labeled polynucleotide, wherein the labeled polynucleotide comprises a first strand having a 5′ terminus and a 3′ terminus and a second strand having a 5′ terminus and a 3′ terminus, and wherein the labeled polynucleotide comprises a double-stranded target sequence between a double-stranded 5′ terminal sequence located proximal to the label, and a double-stranded 3′ terminal sequence located distal to the label, the double-stranded 5′ terminal sequence including the 5′ terminus of the first strand and a sequence adjacent and the 3′ terminus of the second strand and a sequence adjacent, the double-stranded 3′ terminal sequence including the 3′ terminus of the first strand and a sequence adjacent and the 5′ terminus of the second strand and a sequence adjacent;
b) immobilizing the labeled polynucleotide, said immobilizing comprising attaching the 5′ end of the double-stranded 5′ terminal sequence on the first strand to a solid phase binding partner or, in the alternative, attaching the 3′ end of the double-stranded 5′ terminal sequence on the second strand to a solid phase binding partner;
c) introducing a nick or gap at the 5′ end of the double-stranded 5′ terminal sequence on the first strand proximal to the solid phase binding partner;
d) introducing nuclease-resistant bases at the 5′ end of the double-stranded 5′ terminal sequence on the first strand proximal to the solid phase binding partner, thereby creating a non-degradable break in the 5′ end of the double-stranded 5′ terminal sequence on the first strand proximal to the solid phase binding partner; and
e) contacting the labeled polynucleotide with an enzyme with 5′-3′ exonuclease activity under conditions wherein the second strand of the labeled polynucleotide is degraded up to a non-degradable location, at which point the desired polynucleotide is separated from the labeled polynucleotide.

100. A method of preparing a desired polynucleotide comprising the steps of:
a) preparing a labeled polynucleotide, wherein the labeled polynucleotide comprises a first strand having a 5′ terminus and a 3′ terminus and a second strand having a 5′ terminus and a 3′ terminus, and wherein the labeled polynucleotide comprises a double-stranded target sequence between a double-stranded 5′ terminal sequence located proximal to the label, and a double-stranded 3′ terminal sequence located distal to the label, the double-stranded 5′ terminal sequence including the 5′ terminus of the first strand and a sequence adjacent and the 3′ terminus of the second strand and a sequence adjacent, the double-stranded 3′ terminal sequence including the 3′ terminus of the first strand and a sequence adjacent and the 5′ terminus of the second strand and a sequence adjacent;
b) immobilizing the labeled polynucleotide, said immobilizing comprising attaching the 5′ end of the double-stranded 5′ terminal sequence on the first strand to a solid phase binding partner or, in the alternative, attaching the 3′ end of the double-stranded 5′ terminal sequence on the second strand to a solid phase binding partner;
c) introducing nick or gap at the 5′ end of the double-stranded 5′ terminal sequence on the first strand proximal to the solid phase binding partner; and
d) contacting the dsDNA fragment with an enzyme with 5′-3′ exonuclease activity under conditions wherein the first strand and the second strand of the labeled polynucleotide are degraded up to an enzyme collision location, at which point the desired polynucleotide is separated from the labeled polynucleotide.

101. A method of preparing a desired polynucleotide comprising the steps of:
a) preparing a labeled polynucleotide, wherein the labeled polynucleotide comprises a first strand having a 5′ terminus and a 3′ terminus and a second strand having a 5′ terminus and a 3′ terminus, and wherein the labeled polynucleotide comprises a double-stranded target sequence between a double-stranded 5′ terminal sequence located proximal to the label, and a double-stranded 3′ terminal sequence located distal to the label, the double-stranded 5′ terminal sequence including the 5′ terminus of the first strand and a sequence adjacent and the 3′ terminus of the second strand and a sequence adjacent, the double-stranded 3′ terminal sequence including the 3′ terminus of the first strand and a sequence adjacent and the 5′ terminus of the second strand and a sequence adjacent;
b) immobilizing the labeled polynucleotide, said immobilizing comprising attaching the 5′ end of the double-stranded 5′ terminal sequence on the first strand to a solid phase binding partner or, in the alternative, attaching the 3′ end of the double-stranded 5′ terminal sequence on the second strand to a solid phase binding partner;
c) introducing nick or gap at the 3′ end of the double-stranded 5′ terminal sequence on the second strand proximal to the solid phase binding partner;
d) introducing nuclease-resistant bases at the 3′ end of the double-stranded 5′ terminal sequence on the second strand proximal to the solid phase binding partner, thereby creating a non-degradable break in the 3′ end of the double-stranded 5′ terminal sequence on the second strand proximal to the solid phase binding partner; and
e) contacting the dsDNA fragment with an enzyme with 3′-5′ exonuclease activity under conditions wherein the first strand of the labeled polynucleotide is degraded up to a non-degradable location, at which point the desired polynucleotide is separated from the labeled polynucleotide.

102. A method of preparing a desired polynucleotide comprising the steps of:
a) preparing a labeled polynucleotide, wherein the labeled polynucleotide comprises a first strand having a 5′ terminus and a 3′ terminus and a second strand having a 5′ terminus and a 3′ terminus, and wherein the labeled polynucleotide comprises a double-stranded target sequence between a double-stranded 5′ terminal sequence located proximal to the label, and a double-stranded 3′ terminal sequence located distal to the label, the double-stranded 5′ terminal sequence including the 5′ terminus of the first strand and a sequence adjacent and the 3′ terminus of the second strand and a sequence adjacent, the double-stranded 3′ terminal sequence including the 3′ terminus of the first strand and a sequence adjacent and the 5′ terminus of the second strand and a sequence adjacent;
b) immobilizing the labeled polynucleotide, said immobilizing comprising attaching the 5′ end of the double-stranded 5′ terminal sequence on the first strand to a solid phase binding partner or, in the alternative, attaching the 3′ end of the double-stranded 5′ terminal sequence on the second strand to a solid phase binding partner;
c) introducing nick or gap at the 3′ end of the double-stranded 5′ terminal sequence on the second strand proximal to the solid phase binding partner; and
d) contacting the dsDNA fragment with an enzyme with 3′-5′ exonuclease activity under conditions wherein the first strand and the second strand of the labeled polynucleotide are degraded up to an enzyme collision location, at which point the desired polynucleotide is separated from the labeled polynucleotide.

103. (canceled)

104. A method of preparing a desired polynucleotide comprising the steps of:
a) preparing a labeled polynucleotide, wherein the labeled polynucleotide comprises a first strand having a 5′ terminus and a 3′ terminus and a second strand having a 5′ terminus and a 3′ terminus, and wherein the labeled polynucleotide comprises a double-stranded target sequence between a double-stranded 5′ terminal sequence located proximal to the label, and a double-stranded 3′ terminal sequence located distal to the label, the double-stranded 5′ terminal sequence including the 5′ terminus of the first strand and a sequence adjacent and the 3′ terminus of the second strand and a sequence adjacent, the double-stranded 3′ terminal sequence including the 3′ terminus of the first strand and a sequence adjacent and the 5′ terminus of the second strand and a sequence adjacent;
b) immobilizing the labeled polynucleotide, said immobilizing comprising attaching the 5′ end of the double-stranded 5′ terminal sequence on the first strand to a solid phase binding partner or, in the alternative, attaching the 3′ end of the double-stranded 5′ terminal sequence on the second strand to a solid phase binding partner;
c) introducing a 5′ single-stranded overhang at the second strand of the double-stranded 3′ terminal sequence distal to the label; and
d) contacting the single-stranded overhang with an enzyme with 5′-3′ helicase activity under conditions wherein the first strand of the labeled polynucleotide is separated from the second strand up to the end proximal to the solid phase binding partner, at which point the desired polynucleotide is separated from the labeled polynucleotide.

105. A method of preparing a desired polynucleotide comprising the steps of:
a) preparing a labeled polynucleotide, wherein the labeled polynucleotide comprises a first strand having a 5′ terminus and a 3′ terminus and a second strand having a 5′ terminus and a 3′ terminus, and wherein the labeled polynucleotide comprises a double-stranded target sequence between a double-stranded 5′ terminal sequence located proximal to the label, and a double-stranded 3′ terminal sequence located distal to the label, the double-stranded 5′ terminal sequence including the 5′ terminus of the first strand and a sequence adjacent and the 3′ terminus of the second strand and a sequence adjacent, the double-stranded 3′ terminal sequence including the 3′ terminus of the first strand and a sequence adjacent and the 5′ terminus of the second strand and a sequence adjacent;
b) immobilizing the labeled polynucleotide, said immobilizing comprising attaching the 5′ end of the double-stranded 5′ terminal sequence on the first strand to a solid phase binding partner or, in the alternative, attaching the 3′ end of the double-stranded 5′ terminal sequence on the second strand to a solid phase binding partner;
c) introducing a 3′ single-stranded overhang at the first strand of the double-stranded 3′ terminal sequence distal to the label; and
d) contacting the single-stranded overhang with an enzyme with 3′-5′ helicase activity under conditions wherein the first strand of the labeled polynucleotide is separated from the second strand up to the end proximal to the solid phase binding partner, at which point the desired polynucleotide is separated from the labeled polynucleotide.

106. A method of preparing a desired polynucleotide comprising the steps of:
a) preparing a labeled polynucleotide, wherein the labeled polynucleotide comprises a first strand having a 5′ terminus and a 3′ terminus, and wherein the labeled polynucleotide comprises target sequence between a double-stranded 5′ terminal sequence located proximal to the label, and a 3′ terminal sequence located distal to the label, the double-stranded 5′ terminal sequence including the 5′ terminus of the first strand and a sequence adjacent which includes one or more RNA bases, the 3′ terminal sequence including the 3′ terminus of the first strand and a sequence adjacent;
b) immobilizing the labeled polynucleotide, said immobilizing comprising attaching the 5′ end of the double-stranded 5′ terminal sequence on the first strand to a solid phase binding partner;
c) hybridizing a first target-specific primer to the target sequence;
d) contacting the labeled polynucleotide and hybridized primer with an enzyme with polymerase activity under conditions wherein a second strand is synthesized, thereby creating a partially double-stranded fragment;
e) introducing a nick or gap in the partially double-stranded fragment at the 5′ end of the double-stranded 5′ terminal sequence on the first strand proximal to the solid phase binding partner at the site of the one or more RNA bases;
f) introducing one dideoxynucleotide in the nick or gap of step e), thereby creating a non-extendable break in the 5′ end of the double-stranded 5′ terminal sequence on the first strand proximal to the solid phase binding partner;
g) removing any enzymes and unincorporated dideoxynucleotides from step f);
h) hybridizing a second primer to a sequence upstream of the first target-specific primer, said second primer selected fro the group consisting of: (i) a second target-specific primer that hybridizes to a sequence on the target sequence; and (ii) a universal primer that hybridizes to a sequence on the 3′ terminal sequence; and
i) contacting the labeled polynucleotide and hybridized second primer with an enzyme with exonuclease and polymerase activity or, in the alternative, with an enzyme with polymerase-strand-displacement activity, under conditions wherein a new second strand is synthesized up to a non-extendable location, at which point the desired polynucleotide is separated from the labeled polynucleotide.

107. A method of preparing a desired polynucleotide comprising the steps of:
a) preparing a labeled polynucleotide, wherein the labeled polynucleotide comprises a first strand having a 5′ terminus and a 3′ terminus, and wherein the labeled polynucleotide comprises target sequence between a double-stranded 5′ terminal sequence located proximal to the label, and a 3′ terminal sequence located distal to the label, the double-stranded 5′ terminal sequence including the 5′ terminus of the first strand that contains 5 or more RNA bases, the 3′ terminal sequence including the 3′ terminus of the first strand and a sequence adjacent;
b) hybridizing a first target-specific primer to the target sequence;
c) contacting the labeled polynucleotide and hybridized primer with an enzyme with polymerase activity under conditions wherein a second strand is synthesized, thereby creating a partially double-stranded fragment;
d) removing any enzymes and unincorporated deoxynucleotides from step c);
e) introducing a break at the 5′ end of the double-stranded 5′ terminal sequence on the first strand proximal to the solid phase binding partner by removing the 5 or more RNA bases;
f) hybridizing labeled probe in the break introduced in step e), wherein said probe is blocked from extension on the 3′ end and is attached to a solid support, thereby immobilizing the labeled polynucleotide;
g) hybridizing a second primer to a sequence upstream of the first target-specific primer, said second primer selected fro the group consisting of: (i) a second target-specific primer that hybridizes to a sequence on the target sequence; and (ii) a universal primer that hybridizes to a sequence on the 3′ terminal sequence; and
h) contacting the labeled polynucleotide and hybridized second primer with an enzyme with exonuclease and polymerase activity under conditions wherein a new second strand is synthesized up to a non-extendable location, at which point the desired polynucleotide is separated from the labeled polynucleotide.

108. A method of preparing a desired polynucleotide comprising the steps of:
a) preparing a polynucleotide, wherein the polynucleotide comprises a first strand having a 5′ terminus and a 3′ terminus;
b) hybridizing a labeled probe to the 5′ end of the first strand, wherein said probe is attached to a solid support, thereby immobilizing the labeled polynucleotide;
c) hybridizing a target-specific primer to a sequence on the first strand that is 3′ to the hybridized probe; and
d) contacting the labeled polynucleotide and hybridized primer with an enzyme with polymerase activity under conditions wherein a second strand is synthesized, thereby creating a partially double-stranded fragment that is separated from the probe.

109. A method of preparing a desired polynucleotide comprising the steps of:
a) preparing a polynucleotide, wherein the polynucleotide comprises a first strand having a 5′ terminus and a 3′ terminus;
b) hybridizing a target-specific, labeled probe to a sequence on the first strand, wherein said probe is attached to a solid support, thereby immobilizing the labeled polynucleotide;
c) hybridizing a target-specific primer to a sequence on the first strand that is 3′ to the hybridized probe; and
d) contacting the labeled polynucleotide and hybridized primer with an enzyme with polymerase activity under conditions wherein a second strand is synthesized, thereby creating a partially double-stranded fragment that is separated from the probe.
